SEQUENCE LISTING (13 sequences)

SEQ ID NO: 1 - protein, 256 aa, Artificial sequence
  Engineered fusion protein
MSYYHHHHHHDYDSATHIKFSKRDEDGKELAGATMELRDSSGKTISTWISDGQVKDFYLYPGKYTFVETAAPDGYEVATAITFTVNEQGQVTVNGKATKGDAHIGSPANLKALEAQKQKEQRQAAEELANAKKLKEQLEKGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 2 - protein, 504 aa, Artificial sequence
  Engineered fusion protein
MNNNDLFQASRRRFLAQLGGLTVAGMLGPSLLTPRRASMNNNDLFQASRRRFLAQLGGLTVAGMLGPSLLTPRRASMNNNDLFQASRRRFLAQLGGLTVAGMLGPSLLTPRRASAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKGSGSGSAHIVMVDAYKPTK

SEQ ID NO: 3 - protein, 137 aa, Artificial sequence
  Engineered fusion protein
MGLLDLKSMIEKVTGKNALTNYGFYGCYCGWGGRGTPKDGTDWCCWAHDHCYGRLEEKGCNIRTQSYKYRFAWGVVTCEPGPFCHVNLCACDRKLVYCLKRNLRSYNPQYQYFPNILCSGTGSGSKLGDIEFIKVNK

SEQ ID NO: 4 - protein, 138 aa, Artificial sequence
  Engineered fusion protein
MGLLDLKSMIEKVTGKNALTNYGFYGCYCGWGGRGTPKDGTDWCCWAHDHCYGRLEEKGCNIRTQSYKYRFAWGVVTCEPGPFCHVNLCACDRKLVYCLKRNLRSYNPQYQYFPNILCSGTGSGSAHIVMVDAYKPTK

SEQ ID NO: 5 - protein, 503 aa, Artificial sequence
  Engineered fusion protein
MNNNDLFQASRRRFLAQLGGLTVAGMLGPSLLTPRRASMNNNDLFQASRRRFLAQLGGLTVAGMLGPSLLTPRRASMNNNDLFQASRRRFLAQLGGLTVAGMLGPSLLTPRRASAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKGSGSGSATHIKFSKRDGY

SEQ ID NO: 6 - protein, 108 aa, Streptococcus pyogenes
MSYYHHHHHHDYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 7 - protein, 305 aa, Artificial sequence
  Engineered fusion protein
MGSSHHHHHHSSGLVPRGSHMGKLGDIEFIKVNKGSGESGSGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYKGSGEGSGSGSGSAHIVMVDAYKPTK

SEQ ID NO: 8 - protein, 245 aa, Artificial sequence
  Engineered fusion protein
MKYLLPTAAAGLLLLAAQPAMAMVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGSGGTGDSATHIKFSKRDEDGKELAGATMELRDSSGKTISTWISDGQVKDFYLYPGKYTFVETAAPDGYEVATAITFTVNEQGQVTVNGKATKGDAHIGSGHHHHHH

SEQ ID NO: 9 - protein, 245 aa, Artificial sequence
  Engineered fusion protein
MKYLLPTAAAGLLLLAAQPAMAMVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGSGGTGDSATHIKFSKRDEDGKELAGATMELRDSSGKTISTWISDGQVKDFYLYPGKYTFVQTAAPDGYEVATAITFTVNEQGQVTVNGKATKGDAHIGSGHHHHHH

SEQ ID NO: 10 - protein, 251 aa, Aequorea victoria
MSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKKLAAALEHHHHHH

SEQ ID NO: 11 - protein, 251 aa, Artificial sequence
  Engineered fusion protein
MGKGSGSVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNDAQAPKGSVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGSLEHHHHHH

SEQ ID NO: 12 - protein, 642 aa, Artificial sequence
  Engineered fusion protein
MGNAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDSLEGSGSGTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEKLAAAGTGSGEGSGSVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGSLEHHHHHH

SEQ ID NO: 13 - protein, 503 aa, Artificial sequence
  Engineered fusion protein
MNNNDLFQASRRRFLAQLGGLTVAGMLGPSLLTPRRASMNNNDLFQASRRRFLAQLGGLTVAGMLGPSLLTPRRASMNNNDLFQASRRRFLAQLGGLTVAGMLGPSLLTPRRASAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITKGSGSGSKLGDIEFIKVNK

(19) United States
(12) Patent Application Publication
(10) Pub. No.: US 2022/0064691 A1
(43) Pub. Date: Mar. 3, 2022

(54) DECORATED INCLUSION BODY AND USES THEREOF

(71) Applicant: Abera Bioscience AB, Solna (SE)

(72) Inventors: Joen LUIRINK, Solna (SE); Wouter Simon Petrus Jong, Solna (SE); Hendrik Bart VAN DEN BERG VAN SAPAROEA, Solna (SE)

(21) Appl. No.: 17/310,536

(22) PCT Filed: Feb. 14, 2020
(86) PCT No.: PCT/EP2020/053933

§ 371 Date: Aug. 9, 2021

(30) Foreign Application Priority Data
Feb. 15, 2019 (EP) 19157490.4

(51) Int. Cl. C12P 21/02; C07K 14/31; C12N 15/62

(52) U.S. Cl. CPC C12P 21/02; A61K 38/00; C12N 15/625; C07K 14/31

(57) ABSTRACT

The present disclosure relates in general to the field of inclusion bodies. Provided are inclusion bodies comprising a coupling peptide suitable for coupling to a partner peptide through the formation of a covalent isopeptide bond, as well as the use of different ligation systems for enabling efficient and stable decoration of inclusion bodies with, for example, biologically functional molecules to improve the use of inclusion bodies in biotechnology and biomedicine.

### FIELD OF THE INVENTION

The present disclosure relates in general to the field of inclusion bodies. More specifically, the disclosure relates to inclusion bodies comprising a coupling peptide suitable for coupling to a partner peptide through the formation of a covalent isopeptide bond. The present disclosure also relates to the use of different ligation systems for enabling efficient and stable decoration of inclusion bodies with, for example, biologically functional molecules to improve the use of inclusion bodies in biotechnology and biomedicine.

### BACKGROUND OF THE INVENTION

Inclusion bodies (IBs) are generally known as large water-insoluble aggregates that may form upon overproduction of proteins in host cells such as bacterial cells, yeast cells or mammalian cells. Inclusion bodies may be produced upon recombinant protein expression in the cytosol of bacterial cells such as Escherichia coli, and are often regarded as unwanted byproducts of industrial protein production. However, protein expression in IBs has proven to be an effective strategy to avoid some of the problems associated with expression of recombinant proteins in a soluble form. IB expressed proteins are largely resistant to degradation by host cell proteases and less likely to exert toxic effects. Moreover, due to their high buoyant density, IBs are easy to isolate from cell lysates by differential centrifugation, providing fast, robust, and hence cost-efficient protocols for obtaining large amounts of relatively pure protein.

Unfortunately, the propensity of heterologous proteins to form IBs is variable and difficult to predict. Fusing a protein of interest (POI) to an aggregation prone polypeptide or IB formation sequence (IBFS) is a useful strategy to produce it in an insoluble form. Examples of IB forming sequences include ssTorA (Jong et al. 2017 Microb Cell Fact 16:50), TrpΔLE (Derynck et al. 1984 Cell 38:287-97), ketosteroid isomerase (Kuliopulos & Walsh 1994 J Am Chem Soc 116:4599-607), β-galactosidase (Schellenberger et al. 1993 Int J Peptide Protein Res 41:326-32), PagP (Hwang et al. 2012 Protein Expr Purif 85:148-51), EDDIE (Achmuller et al. 2007 Nat Methods 4:1037-43), ELK16 (Wu et al. 2011 Microb Cell Fact 10:9), GFIL8 (Wang et al. 2015 Microb Cell Fact 14:88), PaP3.30 (Rao et al. 2004 Protein Expr Purif 36:11-8), TAF12-HFD (Vidovic et al. 2009, J Pept Sci 15:278-84) and the F4 fragment of PurF (Lee et al. 2000 Biochem Biophys Res Commun 277:575-80).

IBs are very stable and show significant resistance to solubilization by mild detergents (e.g. Triton X-100) and chaotropes (e.g. urea and guanidine hydrochloride). For long, IBs were thought to comprise disordered aggregates formed by non-specific interactions of exposed hydrophobic surfaces. However, evidence from the last decade shows that IBs display ordered amyloid-like structures with proteins accumulating in tightly-packed cross-β configurations (De Groot et al. 2009 Trends Biochem Sci 34:408-16; Wang 2009 Prion 3:139-45). Researchers have also found that IBs often, at least partly, consist of properly folded and biologically active protein (Garcia-Fruitos et al. 2005 Microb Cell Fact 4:27; Jevsevar et al. 2005 Biotechnol Prog 21:632-639). Therefore, rather than being considered waste products of protein production, IBs are nowadays regarded as functional nanoparticles with various potential applications in biotechnology and biomedicine (Rinas et al. 2017 Trends Biochem Sci 42(9):726-737).

Enzymes expressed in the form of IBs, such as reductases, kinases, phosphorylases, lyases and lipases, have been tested as immobilized catalysts with encouraging results (Hrabarova et al. 2015 Insoluble Prot Methods Protoc 1258, 411-422; Garcia-Fruitos, & Villaverde 2010 Korean J Chem Eng 27, 385-389). In biomedicine, IBs have been studied as stimulators of cell proliferation and tissue regeneration (Seras-Franzoso et al. 2015 Nanomedicine 10: 873-891). Moreover, due to their particulate nature and high cell-membrane avidity, IBs are readily internalized by mammalian cells and, therefore, excellent vehicles for intracellular delivery and release of bioactive therapeutic proteins (Vazquez et al. 2012 Adv Mater 24: 1742-1747; Unzueta et al. 2017 Nanotechnology 28:015102; Cespedes et al. 2016 Sci Rep 6: 35765). Several studies have also analyzed the potential of IBs for immunization, such as for inducing antibodies for biochemistry research purposes in rabbits (see, e.g., Cameron et al. 1998 Infect Immun 66(12):5763-70). Moreover, antigenic polypeptide sequences produced as IBs have been tested in vaccination studies and shown to be capable of inducing (protective) immunological responses in various animal species (including mice, calf, lamb, fish and chicken) upon administration via different routes (e.g. oral, intranasal, water immersion) (Yang et al. 2011 Afr Journal Biotechnol 10(41): 8146-8150; Kesik et al. 2007 Vaccine 25: 3619-3628; Kesik et al. 2004 Immunology Letters 9: 197-204; Rivera & Espino 2016 Experimental Parasitology 160: 31-38; Wedrychowicz et al. 2007 Veterinary Parasitology 147: 77-88; PCT application WO2014/052378).

There are also studies exploring the targeting of IBs. Unzueta and co-authors describe the genetic fusion of two homing peptides (ligands R9 and T22) to IB-forming proteins to facilitate targeting IBs to a cell-surface receptor (CXCR4) relevant in cancer therapy (Unzueta et al. 2017, supra). A similar study has also been presented by Jiang et al. FASEB J 2018 Oct. 15. Along the same lines, IBs were designed for specific targeting to CD44⁺ cells through display of genetically fused CD44-binding peptides (Pesarrodona et al. 2016. Biofabrication, 8(2):025001). However, this approach only works for targets and applications for which homing peptides are available, the catalogue of which is still rather limited. In another study, Nahalka and colleagues describe the conjugation of glycoprotein fetuin and non-glycosylated controls with prototype IBs to permit targeting to bacterial adhesins (Talafova et al. 2013 Microbial Cell Factories 12:16). For conjugation, the chemical reagent glutaraldehyde was used as an amine-reactive homobifunctional crosslinker. However, reagents like formaldehyde and glutaraldehyde have a high reactivity towards proteins and are known to interfere with the functionality of proteins. In fact, these are commonly known cellular and protein fixatives and, for example, used for the inactivation of the Bordetella pertussis toxin in an acellular pertussis vaccine (U.S. Pat. No. 5,578,308). Hence, such chemical crosslinking methods are difficult to reconcile with partner proteins that need to remain biologically active upon their coupling to IBs. Moreover, methods involving chemical coupling are often incompatible with industrial scale production of proteins in a cost-efficient manner.

In yet another approach, peptide-peptide interactions between leucine zipper peptide pairs were employed to attach functional partner proteins to inclusion bodies. However, this approach is of limited use as the IB forming protein genetically fused to a leucine zipper peptide must be co-expressed in the same host cell with a functional partner protein genetically fused to a cognate anti-parallel leucine zipper peptide (Steinmann et al. 2010 Appl Environ Microbiol 76(16):5563-9; Choi et al. 2014 PLoS One 9(6): e97093; Han et al. 2017 Metab Eng 40:41-49). Another disadvantage with this co-expression methodology is that conjugation of molecules of non-proteinaceous origin to IBs cannot be achieved.

Moreover, given that leucine zippers associate by protein-protein interactions, IBs produced this way often face stability issues upon administration to humans or animals, or during (long-term) storage, making the use of leucine zipper pairs an unattractive option for attaching molecules to IBs.

In conclusion, a universal method that allows easy and stable decoration of IBs with molecules while maintaining biological functionality would be of great value as it would significantly improve the use of IBs in biotechnology and biomedicine.

### SUMMARY OF THE INVENTION

It is an object of the present invention to overcome the above problems and provide an inclusion body (IB) which can be easily decorated with additional moieties and biologically functional molecules to improve the use of IBs in biotechnology and biomedicine.

According to a first aspect, this and other objects are achieved following the inventors' surprising discovery and production of an inclusion body comprising a coupling peptide suitable for coupling to a partner peptide through the formation of a covalent isopeptide bond.

Suitably, said coupling peptide comprises one residue involved in said isopeptide bond and said partner peptide comprises the other residue involved in said isopeptide bond.

In one embodiment, when said coupling peptide comprises a reactive lysine residue, said partner peptide comprises a reactive asparagine, aspartic acid, glutamine or glutamic acid residue, or when said coupling peptide comprises a reactive asparagine, aspartic acid, glutamine or glutamic acid residue, said partner peptide comprises a reactive lysine residue or a reactive alpha-amino terminus.

In another embodiment, said coupling peptide comprises a reactive asparagine residue and said partner peptide comprises a reactive lysine residue, or said coupling peptide comprises a reactive lysine residue and said partner peptide comprises a reactive asparagine residue.

In one embodiment, said coupling peptide and partner peptide are derived from a protein of a Gram positive or Gram negative bacterium. Suitably, said protein is of a Gram positive bacterium from the Streptococcaceae family, such as Streptococcus pyogenes, Streptococcus pneumoniae or Streptococcus dysgalactiae. Thus, said protein may be adhesin RrgA of Streptococcus pneumoniae, fibronectin-binding protein FbaB of Streptococcus pyogenes, major pilin protein Spy0128 of Streptococcus pyogenes, or fibronectin-binding protein CnaB of Streptococcus dysgalactiae, or a protein with at least 70% sequence identity thereto which is capable of forming one or more isopeptide bonds.

In one embodiment, the coupling peptide is selected from the group consisting of SpyTag, KTag, SnoopTag, SpyTag002, SpyTag003, SpyTag0128, SdyTag, DogTag, SnoopTagJr and BDTag.

In a particular embodiment, the coupling peptide is selected from the group consisting of SpyTag, KTag, SnoopTag, SpyTag002, SpyTag0128, SdyTag, DogTag and SnoopTagJr.

In one embodiment, the partner peptide is selected from the group consisting of SpyTag, KTag, SpyCatcher, SnoopCatcher, SpyCatcher002, SpyCatcher003, SpyCatcher0128, SdyCatcher, DogTag, SnoopTagJr and BDTag.

In a particular embodiment, the partner peptide is selected from the group consisting of SpyTag, KTag, SpyCatcher, SnoopCatcher, SpyCatcher002, SpyCatcher0128, SdyCatcher, DogTag and SnoopTagJr.

In one embodiment, there is provided an inclusion body according to the first aspect, wherein the coupling peptide and partner peptide form a ligation pair selected from the group consisting of SpyTag-SpyCatcher, SpyTag-SpyCatcher002, SnoopTag-SnoopCatcher, SpyTag002-SpyCatcher002, SpyTag002-SpyCatcher, SpyTag003-SpyCatcher003, SpyTag0128-SpyCatcher0128, SdyTag-SdyCatcher, KTag-SpyTag, SpyTag-KTag, DogTag-SnoopTagJr, SnoopTagJr-DogTag, SpyTag-BDTag and BDTag-SpyTag.

In a particular embodiment, there is provided an inclusion body according to the first aspect, wherein the coupling peptide and partner peptide form a ligation pair selected from the group consisting of SpyTag-SpyCatcher, SpyTag-SpyCatcher002, SnoopTag-SnoopCatcher, SpyTag002-SpyCatcher002, SpyTag002-SpyCatcher, SpyTag0128-SpyCatcher0128, SdyTag-SdyCatcher, KTag-SpyTag, SpyTag-KTag, DogTag-SnoopTagJr and SnoopTagJr-DogTag.

In another embodiment, there is provided an inclusion body according to the first aspect, wherein (i) the coupling peptide is KTag, the partner peptide is SpyTag and the formation of a covalent isopeptide bond is mediated by addition of SpyLigase; (ii) the coupling peptide is KTag, the partner peptide is SpyTag002 and the formation of a covalent isopeptide bond is mediated by addition of SpyLigase; (iii) the coupling peptide is SpyTag, the partner peptide is KTag and the formation of a covalent isopeptide bond is mediated by addition of SpyLigase; (iv) the coupling peptide is SpyTag002, the partner peptide is KTag and the formation of a covalent isopeptide bond is mediated by addition of SpyLigase; (v) the coupling peptide is DogTag, the partner peptide is SnoopTagJr and the formation of a covalent isopeptide bond is mediated by addition of SnoopLigase; (vi) the coupling peptide is SnoopTagJr, the partner peptide is DogTag and the formation of a covalent isopeptide bond is mediated by addition of SnoopLigase; (vii) the coupling peptide is SpyTag, the partner peptide is BDTag and the formation of a covalent isopeptide bond is mediated by addition of SpyStapler; or (viii) the coupling peptide is BDTag, the partner peptide is SpyTag and the formation of a covalent isopeptide bond is mediated by addition of SpyStapler.

In a particular embodiment, there is provided an inclusion body according to the first aspect, wherein (i) the coupling peptide is KTag, the partner peptide is SpyTag and the formation of a covalent isopeptide bond is mediated by addition of SpyLigase; (ii) the coupling peptide is KTag, the partner peptide is SpyTag002 and the formation of a covalent isopeptide bond is mediated by addition of SpyLigase; (iii) the coupling peptide is SpyTag, the partner peptide is KTag and the formation of a covalent isopeptide bond is mediated by addition of SpyLigase; (iv) the coupling peptide is SpyTag002, the partner peptide is KTag and the formation of a covalent isopeptide bond is mediated by addition of SpyLigase; (v) the coupling peptide is DogTag, the partner peptide is SnoopTagJr and the formation of a covalent isopeptide bond is mediated by addition of SnoopLigase; or (vi) the coupling peptide is SnoopTagJr, the partner peptide is DogTag and the formation of a covalent isopeptide bond is mediated by addition of SnoopLigase.

According to a second aspect of the invention, there is provided a complex comprising the inclusion body according to the first aspect coupled to the partner peptide via a covalent isopeptide bond between the coupling peptide and the partner peptide.

In one broad embodiment, the inclusion body according to the first aspect, or the complex according to the second aspect further comprises at least one protein of interest (POI), or a portion thereof.

In one embodiment, said protein of interest is a protein with a therapeutic purpose that treats a condition or disorder selected from the group consisting of cancer, autoimmune disease, inflammatory disease, transplant rejection and infectious disease.

In another embodiment, said protein of interest is a protein with a prophylactic purpose that protects against a condition or disorder selected from the group consisting of cancer, autoimmune disease, inflammatory disease, transplant rejection and infectious disease.

Preferably, said protein of interest is an antigen or a fragment thereof.

Said antigen may be selected from the group consisting of an antigen from an infectious organism, a tumor antigen, a tumor stroma antigen and a tumor associated antigen.

In some embodiments, the inclusion body according to the first aspect further comprises an inclusion body forming sequence (IBFS). Said IBFS may for example be selected from ssTorA, TrpΔLE, ketosteroid isomerase, β-galactosidase, PagP, EDDIE, ELK16, GFIL8, PaP3.30, TAF12-HFD and the F4 fragment of PurF. Other IBFS:s, suitable for use in the present invention, are described in WO2018/138316.

In one broad embodiment, there is provided an inclusion body according to the first aspect, or a complex according to the second aspect, wherein the partner peptide comprises an additional moiety.

The additional moiety may for example be selected from the group consisting of a glycan, an adhesion molecule, an enzyme and a traceable probe.

In one embodiment, the additional moiety is an immune modulating compound. Said immune modulating compound may for example be selected from the group consisting of a cytokine, an adjuvant, an antibody, a Nanobody® molecule, a DARPIN, PAMP, a TLR ligand or agonist, RNA, DNA, an immunomodulating peptide, a peptidomimetic, a T helper cell epitope, an immune checkpoint inhibitor, PLGA, chitosan and TRAIL.

In one embodiment, the additional moiety is a targeting moiety. Said targeting moiety may have an affinity for a cell of the immune system. Thus, said targeting moiety may have an affinity for a surface exposed component of a cell of the immune system. The surface exposed component may be selected from the group consisting of CD4, CD8, CD1, CD180, IgA, IgD, IgE, IgG, IgM, TCR, CRDs, Toll-like receptors (TLRs), nucleotide-binding oligomerization domain-like receptors (NLRs), retinoic acid-inducible gene I-like helicases receptors (RLRs), and C-type lectin receptors (CLRs), endocytic receptors, CD205/DEC205, CD209/DC-SIGN, Clec9A/DNGR-1/CD370, Clec7A/Dectin-1/CD369, Clec6A/Dectin-2, Clec12A, CD1d, CD11c, CD11 b, CD40, CD152/CTLA-4, CD279/PD-1, NOD-like receptors, RIG-I-like receptors, PRRs, CCRs, CD36, Siglec H, PDCTREM, Langerin, MMR, D-SIGN and folate receptors.

Suitably, said targeting moiety has an affinity for at least one diseased cell. Said diseased cell may be a tumor cell of a cancer selected from the group consisting of lymphoma, leukemia, myeloma, lung cancer, melanoma, renal cell cancer, ovarian cancer, glioblastoma, Merkel cell carcinoma, bladder cancer, head and neck cancer, colorectal cancer, esophageal cancer, cervical cancer, gastric cancer, hepatocellular cancer, prostate cancer, breast cancer, pancreatic cancer and thyroid cancer.

In one embodiment, the targeting moiety is selected from the group consisting of an antibody, an antibody domain and an antibody fragment retaining antibody binding capacity.

According to a third aspect of the invention, there is provided a nucleic acid encoding the inclusion body forming polypeptide of the inclusion body according to the first aspect.

According to a fourth aspect of the invention, there is provided a genetic construct comprising the nucleic acid according to the third aspect.

According to a fifth aspect of the invention, there is provide a host cell comprising the nucleic acid according to the third aspect or the genetic construct according to the fourth aspect.

According to a sixth aspect of the invention, there is provided a composition comprising the inclusion body according to the first aspect, the complex according to the second aspect, the nucleic acid according to the third aspect, the genetic construct according to the fourth aspect, and/or the host cell according to the fifth aspect.

According to a seventh aspect of the invention, there is provided an inclusion body according to the first aspect, a complex according to the second aspect, or a composition according to the sixth aspect for use as a medicament.

According to an eighth aspect of the invention, there is provided an inclusion body according to the first aspect, a complex according to the second aspect, or a composition according to the sixth aspect for use as a diagnostic, prognostic, prophylactic or therapeutic agent.

According to a ninth aspect of the invention, there is provided an inclusion body according to the first aspect, a complex according to the second aspect, or a composition according to the sixth aspect for use as a vaccine.

According to a tenth aspect of the invention, there is provided a method of treatment of a disease or disorder in a subject comprising the step of introducing the inclusion body according to the first aspect, the complex according to the second aspect, or the composition according to the sixth aspect to said subject.

According to an eleventh aspect of the invention, there is provided a method of diagnosis or prognosis of a disease or disorder in a subject using the inclusion body according to the first aspect, the complex according to the second aspect, or the composition according to the sixth aspect.

According to a twelfth aspect of the invention, there is provided a method of vaccination or immunization comprising the step of introducing the inclusion body according to the first aspect, the complex according to the second aspect, or the composition according to the sixth aspect to said subject.

Suitably, said subject is an animal.

In one embodiment, said animal is a mammal selected from a human, a farm animal (e.g. cattle, sheep, pig and goat) and a companion animal (e.g. horse, dog and cat).

In other embodiments, said animal is selected from a bird (e.g. poultry) and a fish (e.g. salmon, trout, seabass, tilapia and catfish).

According to a thirteenth aspect of the invention, there is provided a method of producing the complex according to the second aspect, comprising the step of conjugating the inclusion body of the first aspect to a partner peptide to thereby produce said complex.

Suitably, the complex is produced by the formation of a covalent isopeptide bond between the coupling peptide of the inclusion body and the partner peptide. Typically, the coupling peptide comprises one residue involved in said isopeptide bond and said partner peptide comprises the other residue involved in said isopeptide bond.

In one embodiment, the inclusion body is conjugated to a partner peptide presented in a lysate, such as a cell lysate, such as a bacterial lysate.

In another embodiment, the inclusion body is conjugated to a purified partner peptide.

The coupling peptide may be selected from the group consisting of SpyTag, KTag, SnoopTag, SpyTag002, SpyTag003, SpyTag0128, SdyTag, DogTag, SnoopTagJr and BDTag.

Thus, the coupling peptide may be selected from the group consisting of SpyTag, KTag, SnoopTag, SpyTag002, SpyTag0128, SdyTag, DogTag and SnoopTagJr.

The partner peptide may be selected from the group consisting of SpyTag, KTag, SpyCatcher, SnoopCatcher, SpyCatcher002, SpyCatcher003, SpyCatcher0128, SdyCatcher, DogTag, SnoopTagJr and BDTag.

The partner peptide may, for example, be selected from the group consisting of SpyTag, KTag, SpyCatcher, SnoopCatcher, SpyCatcher002, SpyCatcher0128, SdyCatcher, DogTag and SnoopTagJr.

In particular embodiments, the complex is produced following the formation of a coupling peptide-partner peptide ligation pair selected from the group consisting of SpyTag-SpyCatcher, SpyTag-SpyCatcher002, SnoopTag-SnoopCatcher, SpyTag002-SpyCatcher002, SpyTag002-SpyCatcher, SpyTag003-SpyCatcher003, SpyTag0128-SpyCatcher0128, SdyTag-SdyCatcher, KTag-SpyTag, SpyTag-KTag, DogTag-SnoopTagJr, SnoopTagJr-DogTag, SpyTag-BDTag and BDTag-SpyTag.

Thus, the complex may be produced following the formation of a coupling peptide-partner peptide ligation pair selected from the group consisting of SpyTag-SpyCatcher, SpyTag-SpyCatcher002, SnoopTag-SnoopCatcher, SpyTag002-SpyCatcher002, SpyTag002-SpyCatcher, SpyTag0128-SpyCatcher0128, SdyTag-SdyCatcher, KTag-SpyTag, SpyTag-KTag, DogTag-SnoopTagJr and SnoopTagJr-DogTag.

### BRIEF DESCRIPTION OF THE FIGURES

FIG. 1A. Schematic drawing of an inclusion body decorated with a green fluorescent Nanobody® molecule (GFPnb) with affinity for GFP (SEQ ID NO:10) using the ligation system of SpyTag and SpyCatcher. The inclusion body (IB) is seen expressing SpyTag on its surface, which is covalently bound to SpyCatcher. The additional moiety GFPnb is linked to SpyCatcher and the GFPnb is then non-covalently bound to the GFP (Example 1).

FIG. 1B. Ribbon diagram of the partner peptide (binding protein partner) SpyCatcher (left) and GFP bound to GFPnb (right) (Example 1).

FIG. 2A. SDS-PAGE analysis of successful IB decoration using the SpyTag/SpyCatcher ligation system. Arrow ( [symbol] ) at ˜75 kDa adduct indicates the conjugation of SpyTag to SpyCatcher, linking fusion protein SpyCatcher-SnoopCatcher (SEQ ID NO:1) to ssTorA(3×)-MBP-SpT (SEQ ID NO:2) IBs (Example 2).

FIG. 2B. SDS-PAGE analysis of successful IB decoration using the SnoopTag/SnoopCatcher ligation system. Arrow ( [symbol] ) at ˜75 kDa adduct indicates the conjugation of SnoopTag to SnoopCatcher, linking fusion protein SpyCatcher-SnoopCatcher to ssTorA(3×)-MBP-SnT IBs (Example 2).

FIG. 3A. SDS-PAGE analysis of successful conjugation of Pla2 IBs without inclusion body forming sequence ssTorA, and AEDO IBs with inclusion body forming sequence ssTorA to a partner peptide making use of either the SpyTag/SpyCatcher or SnoopTag/SnoopCatcher system. Adducts are indicated with an arrow ( [symbol] ) (Example 3).

FIG. 3B. Verification of successful conjugation of Pla2 IBs without inclusion body forming sequence ssTorA, and AEDO IBs with inclusion body forming sequence ssTorA to a partner peptide making use of either the SpyTag/SpyCatcher or SnoopTag/SnoopCatcher system through Western blotting using antibodies recognizing polyHistidine detection tag incorporated in the SpyCatcher-SnoopCatcher fusion protein. Adducts are indicated with an arrow ( [symbol] ) (Example 3).

FIG. 4A. Phase contrast and fluorescence microscopy analysis of successful ligation between SnT-mEGFP-SpT (SEQ ID NO:7) and ssTorA(3×)-MBP-KT (SEQ ID NO:5) IBs using tripartite system. GFP-fluorescence signals are emitted by IBs mixed with SnT-mEGFP-SpT and SpyLigase (SEQ ID NO:6) (+SpyLigase), whereas no signals are detected when SpyLigase is absent (-) (Example 4).

FIG. 4B. SDS-PAGE analysis of ssTorA(3×)-MBP-KT IBs and soluble SnT-mEGFP-SpT mixed with and without the presence of SpyLigase (tripartite system). Analysis shows an emerging band symbolizing bond formation between ssTorA(3×)-MBP-KT IBs and SnT-mEGFP-SpyTag when SpyLigase is present (+SpyLigase) and the lack thereof when SpyLigase is absent (-). The adduct of ssTorA(3×)-MBP-KT-SnT-mEGFP-SpT is indicated with an arrow ( [symbol] ) (Example 4).

FIG. 5A. Phase contrast and fluorescence microscopy analysis of successfully decorating IBs with GFP-specific Nanobody® molecules (GFPnb). ssTorA(3×)-MBP-SpT IBs mixed with fusion protein SpC-GFPnb and GFP are seen to emit a signal in the fluorescence microscopy analysis, whereas IBs mixed with GFPnb-SpC EQ (SEQ ID NO:9) (E77Q amino acid substitution in SpyCatcher interfering with isopeptide bond formation) and GFP do not (Example 5).

FIG. 5B. SDS-PAGE analysis showing that mixing of ssTorA(3×)-MBP-SpT IBs with GFPnb-SpC (SEQ ID NO:8) protein gives rise to an adduct whereas mixing of ssTorA(3×)-MBP-SpT IBs with mutant GFPnb-SpC EQ does not. Adduct is indicated by an arrow ( [symbol] ) (Example 5).

FIG. 6A. SDS-PAGE analysis of ssTorA(3×)-AEDO-SpT IBs mixed with a fusion protein comprising a tandem-fused dual version of the antibody-binding domain of protein A, ZZ-domain, and a C-terminally located SpyCatcher002 moiety, SpC2, forming ZZ-SpC2 (SEQ ID NO:11). As a control, ZZ-SpC2 was also mixed with ssTorA(3×)-AEDO lacking SpT. IB-associated heavy chain material and the adduct of ssTorA(3×)-AEDO-SpT bound to ZZ-SpC2 are indicated by an arrow ( [symbol] ) (Example 6).

FIG. 6B. Fluorescence microscopy analysis of ssTorA(3×)-AEDO-IBs, with and without SpT, pre-incubated with ZZ-SpC2 as above and incubated with Alexa 594 Rabbit anti-Mouse IgGs.

FIG. 7. SDS-PAGE analysis of successful coupling of SpC2-equipped ZZ-domain (ZZ) and Protein NG (AG) to SpT-carrying IBs. IB-associated heavy- and light chain material, as well as adducts (ZZ adduct, AG adduct) are indicated by arrows ( [symbol] ) (Example 7).

FIG. 8. SDS-PAGE analysis of successful conjugation of ZZ-SpC2 from bacterial lysate to ssTorA(3×)-AEDO-SpT. Adduct is indicated by an arrow ( [symbol] ) (Example 8).

### DEFINITIONS

As used herein, the following definitions are provided to facilitate the understanding of the present invention.

The term “inclusion body”, sometimes abbreviated “IB”, refers to an insoluble deposit of aggregated polypeptides in the cytoplasm or nucleus of a cell. Herein, the term mainly refers to inclusion bodies formed within the cytoplasm of prokaryotic, bacterial cells. The term may also refer to polypeptide aggregates in the periplasm of prokaryotic, bacterial cells or to polypeptide aggregates in the cytoplasm and/or nucleus of eukaryotic cells. Inclusion bodies may form spontaneously within a host cell, for example as the result of overexpression of insoluble or partly insoluble polypeptides. In the present disclosure, a polypeptide or protein of interest that is normally soluble or partly soluble within a host cell may be fused to an IB forming sequence, resulting in a fusion polypeptide comprising the polypeptide of interest operably linked to the IB forming sequence. When the fusion polypeptide is expressed, the inclusion body forming sequence induces the fusion polypeptide, and thus the polypeptide of interest, to form inclusion bodies. The aggregated polypeptides contained in the inclusion bodies may be misfolded, partly misfolded or may have a native or nearly native fold. The insoluble form of a polypeptide in an inclusion body protects the polypeptide from degradation by proteolytic enzymes within the host cell. Moreover, it protects the host cell from any toxic effect that the polypeptide might have in its soluble, native form. Also, the formation of inclusion bodies may facilitate isolation and purification of certain polypeptides that are otherwise difficult to purify or that otherwise require many and/or expensive purification steps. Means and methods to identify inclusion bodies and quantify inclusion body formation are well known in the art. Non-limiting examples of such means and methods include inclusion body fractionation assay, phase contrast microscopy, other optical measuring techniques, particle size measurements, gel separation assays (e.g. SDS-PAGE), proteolytic digestion and electron microscopy.

Thus, as indicated above, the term “inclusion body forming sequence”, sometimes abbreviated “IBFS” herein, refers to a polypeptide sequence that induces formation of inclusion bodies when fused to a polypeptide of interest. The inclusion body forming sequence causes a fusion polypeptide comprising the polypeptide of interest and a peptide encoded by the inclusion body forming sequence to aggregate in inclusion bodies.

The term “polypeptide” is herein used to designate a series of two or more amino acid residues connected to one another by peptide bonds between the alpha-amino and carboxy groups of adjacent residues. The term is used to designate a peptide of unspecified length. Thus, peptides, oligopeptides, polypeptides and proteins are included within the definition of a “polypeptide” herein. Typically, although not exclusively, the term “peptide” is herein used to designate a short polypeptide, for example having a length of about two amino acids to about 50 amino acids. The term “protein” is herein used to designate longer and/or more complex polypeptides, such as a complex of two or more polypeptide chains. A protein may also be bound to cofactors or other proteins. The terms “peptide”, “polypeptide” and “protein” may also include posttranslational modifications, such as glycosylations, acetylations, phosphorylations etc. Polypeptides comprising one or more amino acid analogue or labeled amino acid are also included within the definition.

The interchangeable terms “polypeptide sequence”, “peptide sequence” and “protein sequence” refer to the order of amino acids in a polypeptide, peptide or protein. As is conventional, a polypeptide sequence is herein generally reported from the N-terminal end to the C-terminal end.

The terms “polypeptide of interest”, “peptide of interest” and “protein of interest”, abbreviated “POI”, are used interchangeably to refer to a polypeptide, peptide or protein that is of interest to a user of the present invention and that may be expressed by the genetic machinery of a host cell, e.g. as a recombinant protein. In some situations, the term “POI” is also to be understood as referring to the genetic sequence encoding the POI in question. The POI can be any type of POI. For example, the POI may be a heterologous or homologous polypeptide, a soluble or partly soluble cytoplasmic polypeptide, a soluble or partly soluble secretory polypeptide or a membrane polypeptide. In some embodiments, the POI is a polypeptide that is toxic to the host cell, that degrades easily in the host cell or that is difficult to purify from the host cell when in soluble form. The POI may comprise translationally fused peptides or fragments derived from various different proteins or of synthetic origin. The POI may be of any length. In particular, the POI may be at least from about 2, 5, 10, 25 or 50 amino acids long, and may be up to 1000, 1500, 2000, 3000 or 5000 amino acids long. In some embodiments, the POI may comprise translationally fused peptides derived from various different proteins or of synthetic origin.

The terms “fusion polypeptide”, “fusion protein” and “fusion peptide” refer to a polymer of amino acids, i.e. a polypeptide, protein or peptide, comprising at least two portions, each portion representing a distinct function and/or origin. A fusion polypeptide of the present invention may comprise, in any order, at least a first portion comprising the disclosed inclusion body forming sequence and at least a second portion comprising a polypeptide or peptide of interest. The fusion polypeptide may in alternative embodiments comprise more than one inclusion body forming sequence and/or more than one POI. The fusion polypeptide may for example comprise one or more inclusion body forming sequences at its N-terminal end and/or one or more inclusion body forming sequences at its C-terminal end. It may also comprise further portions comprising other functionalities, such as a cleavable element for separation of the inclusion body forming sequence(s) from the POI(s).

The term “genetic construct” refers to an engineered combination of genetic elements, such as genes or other polypeptide coding elements, promoters, regulatory elements, transcription and termination regions etc, assembled into a single nucleic acid. A genetic construct may also comprise genetic elements encoding two or more portions from different polypeptides, such that the genetic construct encodes a fusion polypeptide comprising the two or more portions. An expression vector is an example of a genetic construct. Another example of a genetic construct is a polypeptide-coding nucleic acid which is integrated into a genome of a host and expressed therefrom.

The expressions “recombinant polypeptide”, “recombinant genetic construct” and “recombinant complex” refer to polypeptides or nucleic acids that result from the use of laboratory methods to bring together genetic material from multiple sources, creating nucleic acids and polypeptides encoded therefrom that would not otherwise be found in nature.

The terms “host”, “host cell” and “recombinant host cell” are used interchangeably herein to indicate a prokaryotic or eukaryotic cell into which one or more vectors or isolated and purified nucleic acid molecules have been or can be introduced. Thus, the genetic construct may be expressed from a vector or integrated into the genome of the host and expressed therefrom.

In a preferred embodiment, the host cell is a microbial host cell. In another preferred embodiment, the microbial host cell is a bacterial cell. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

The terms “ligation system” or “protein ligation system” are used to describe any system comprising at least a first and a second molecular part which have affinity for each other and between which a bond of some sort may be formed. The ligation system may for example consist of two peptides between which a covalent bond may form spontaneously or with the assistance of an enzyme, or where chemical crosslinking means are used to link different molecules together. In the present disclosure, a ligation system specifically refers to a first and a second peptide that have the functionality to spontaneously form an isopeptide bond or that are able to form an isopeptide bond in the presence of an enzyme (e.g. a ligase, such as SpyLigase, SnoopLigase or SpyStapler). It is further possible to link additional molecules to each of the two peptides, creating a complex wherein it is the two peptides with affinity for each other which constitute the ligate of the ligation system. In the current disclosure, one of the two peptides of the ligation system is named “coupling peptide” and the other peptide of the ligation system is named “partner peptide”, wherein the coupling peptide is linked to an IB and the partner peptide may optionally link an additional molecule to the entire complex. Thus, the term “complex” is used herein to describe an IB comprising a coupling peptide and coupled to the partner peptide via a covalent isopeptide bond between the coupling peptide and the partner peptide.

As mentioned, the term “coupling peptide” is used herein to describe one of the two peptides constituting the ligation system. More specifically, a coupling peptide is a polymer of amino acids having the distinct functionality of binding the other peptide of the ligation system, i.e. the “partner peptide”. In the context of the invention, the coupling peptide is typically genetically fused to a POI (e.g. as a C-terminal, N-terminal or internal peptide tag) and optionally also to an IBFS, forming a genetic construct. Said genetic construct may be incorporated into a vector which is introduced and subsequently expressed in a host cell to produce a recombinant polypeptide comprising an inclusion body with a coupling peptide accessible to the partner peptide of the ligation system. The genetic construct may also be integrated into the genome of the host cell and expressed therefrom.

The term “partner peptide” refers to the other peptide of the ligation system as defined in the context of the invention. It is a polymer of amino acids having the distinct functionality of binding the first part of the ligation system, i.e. the “coupling peptide”. Unlike the coupling peptide, the partner peptide is typically produced unattached to an IB. Optionally, it may be expressed in fusion to or in a complex with an additional molecule. While the parts of the ligation systems described herein are referred to as “peptides”, it will be appreciated that they may, in some embodiments, comprise more than 50 amino acids.

The terms “SpyTag-SpyCatcher ligation system”, “SpyTag” and “SpyCatcher” describe the first part (coupling peptide) and second part (partner peptide), respectively, of a particular ligation system which may be used in the current disclosure. The ligation system is derived from a CnaB domain present in the Streptococcus pyogenes fibronectin-binding protein FbaB. Within the hydrophobic core of this domain, a triad of amino acids (lysine, aspartate and a catalytic glutamate) spontaneously form an isopeptide bond (Hagan et al. 2010 Angew Chem Int Ed Engl November 2; 49(45):8421-5). The isolated CnaB domain was converted into a protein ligation system by splitting it into a peptide, the so called “SpyTag”, sometimes abbreviated “SpT”, and the remaining protein partner called the “SpyCatcher”, sometimes abbreviated “SpC” (Zakeri et al. 2012 Proc Natl Acad Sci USA 109). The two peptides possess the ability to spontaneously form an isopeptide bond between each other. In the context of the invention, the SpyTag and the protein of interest in an IB may form a recombinant polypeptide wherein the SpyTag acts as the coupling peptide and is accessible to the partner peptide (e.g. by being displayed on the surface of the IB). The SpyCatcher acts as the partner peptide and may form a robust bond to the SpyTag.

The term “SnoopTag-SnoopCatcher ligation system”, “SnoopTag” and “SnoopCatcher” refer to the first part (coupling peptide) and second part (partner peptide) respectively of another ligation system, which has been derived from the D4 Ig-like domain of the adhesin RrgA from Streptococcus pneumoniae (Veggiani et al. 2016 Proc Natl Acad Sci USA 113(5):1202-7). To create this system, the D4 Ig-like domain was cleaved to create a coupling peptide called “SnoopTag”, sometimes abbreviated “SnT”, and a remaining partner peptide “SnoopCatcher”, sometimes abbreviated “SnC”. In the context of the invention, the SnoopTag and the protein of interest of an IB may form a recombinant polypeptide, wherein the SnoopTag is accessible to the partner peptide (e.g. by being displayed on the surface of the IB).

The terms “KTag” and “SpyLigase” refer to a peptide tag and an enzyme, respectively. After the successful adaptation of the CnaB domain into the SpyTag/SpyCatcher protein ligation system, the SpyCatcher was further split up into the “KTag”, sometimes abbreviated “KT”, and “SpyLigase” (Fierer et al. 2014 Proc Natl Acad Sci USA April 1; 111(13):E1176-81). KTag acts as a coupling peptide which may form a covalent bond with a partner peptide in the presence of SpyLigase, which is needed to catalyze the bond formation. It has also been discovered that, in the presence of SpyLigase, both KTag and SpyTag may alternate between acting as a coupling peptide and acting as a partner peptide. However, SpyLigase remains a polypeptide separate from the ligation system upon bond formation.

Although the “Catchers” referred to herein are generally referred to as partner peptides, some Catchers may also be suitable as coupling peptides (i.e. moieties expressed in the IB and accessible to the partner peptides).

The term “operably linked” refers to the association of a first portion of a polypeptide or a nucleic acid fragment with a second portion of the polypeptide or nucleic acid fragment, such that the function of one of the portions is affected by the other. For example, a fusion polypeptide according to the invention comprises a POI operably linked to a coupling peptide and optionally operably linked to an IBFS, meaning that the POI is operably linked to and affected by the coupling peptide and optionally by the IBFS, but that the parts are not necessarily contiguously fused. Similarly, with regard to nucleic acids, a promoter may for instance be operably linked to a coding sequence, for example coding for a fusion polypeptide according to the invention, meaning that the promoter is able to affect the expression of the coding sequence, i.e. that the coding sequence is under transcriptional control of the promoter. A translation initiation region such as a ribosome binding site is operably linked to a nucleic acid sequence encoding e.g. a polypeptide, if it is positioned so as to facilitate translation of the polypeptide.

The term “decorated IB” refers to an IB which expresses a coupling peptide and wherein an isopeptide bond has been formed between a portion of the coupling peptide that is accessible to a partner peptide (e.g. a portion displayed on the surface of the IB), and the partner peptide. Said partner peptide may further bind to an additional moiety which may have a certain functionality.

The term “moiety” as used herein refers to a molecular or cellular component, i.e. the term “moiety” is not limited to concern half or part of a molecule as the word has previously been defined and used in the technical field of chemistry.

The term “additional moiety” refers to any molecular or cellular component which can be linked to a partner peptide, for example a peptide, cytokine, adjuvant, antibody, glycan, adjuvant, adhesion, enzyme and traceable probe.

The term “targeting moiety” refers to any molecular or cellular component which can be linked to a partner peptide and which has the functionality of targeting a specific mark, such as for example a target molecule, tissue, cell, receptor or the like.

The term “functional moiety” refers to at least one molecular or cellular component which possess a certain functionality and the term may thus refer to a functional component or group of any kind.

The term “antigen” refers to a molecule which is capable of inducing an immune response in a host organism.

The term “adjuvant” refers to a pharmacological or immunological agent which has the ability to modify the effect of other agents.

The term “immunostimulatory tool” refers to a molecular or cellular component or system of molecular or cellular components which can be used to stimulate the immune system of a host, i.e. to trigger the immune system in such a way that for example an immune response is initiated. This can be done by for example introducing a certain antigen into the host cell wherein the antigen may be part of the immunostimulatory tool.

The term “REDO” refers to “antigenic epitopes of different origin”.

The term “acceptable carrier, diluent or excipient” refers to a solid or liquid filler, diluent or encapsulating substance that may be safely used in local or systemic administration (e.g. of a pharmaceutical composition or a vaccine). Any safe route of administration may be employed, including oral, parenteral, rectal, sublingual, buccal, intravenous, intra-articular, intra-muscular, intra-dermal, subcutaneous, inhalational, intra-ocular, intraperitoneal, intracerebroventricular, topical, mucosal, and transdermal administration. Depending on the particular route of administration, a variety of carriers, diluents and excipients known in the art may be used. These may for example be selected from the group consisting of sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates, water and pyrogen-free water.

### DETAILED DESCRIPTION

The present invention relates generally to an inclusion body which expresses and displays a coupling peptide (e.g. a peptide tag). Through the formation of a covalent isopeptide bond, said coupling peptide may be coupled to a partner peptide optionally fused to an additional moiety, creating a link between the inclusion body and the additional moiety.

Thus, broadly speaking, the present disclosure is based on the inventors' surprising discovery and development of a powerful system and method for decorating inclusion bodies with a variety of functional moieties using different ligation systems.

Several attempts have been made to achieve protein ligation to inclusion bodies. However, as described in the background section, achieving ligation is associated with a number of obstacles and drawbacks. For example, preserving the biological activity of the proteins is a problem encountered when using chemical crosslinking. Unstable bond formation and the limitation to only decorate the inclusion body with other proteins are problems associated with using the protein-protein interactions of the leucine zipper peptide pairs.

The present inventors have surprisingly and advantageously managed to produce an IB with a coupling peptide, which coupling peptide remains functional and retains its binding specificity to its partner peptide. As a result, a very robust covalent isopeptide bond is formed, linking the coupling peptide directly to the partner peptide, thus linking the inclusion body to the partner peptide.

Typically, the coupling peptide comprises one residue involved in the isopeptide bond while the partner peptide comprises the other residue involved in the isopeptide bond. For example, when the coupling peptide comprises a reactive lysine residue, the partner peptide comprises a reactive asparagine, aspartic acid, glutamine or glutamic acid residue, or when the coupling peptide comprises a reactive asparagine, aspartic acid, glutamine or glutamic acid residue, the partner peptide comprises a reactive lysine residue or a reactive alpha-amino terminus. Thus, the coupling peptide may comprise a reactive asparagine residue while the partner peptide may comprise a reactive lysine residue, or the coupling peptide may comprise a reactive lysine residue while the partner peptide may comprise a reactive asparagine residue.

Hence, by combining a ligation system comprising a coupling peptide with affinity for a partner peptide with an IB optionally comprising or constituting a POI, the inventors have created a novel and inventive platform for inclusion body decoration and subsequent targeted antigen and/or drug delivery and other applications.

This combination has been made possible due to the inventors' ability to covalently link proteinaceous moieties to be accessible on IBs, which provides the IBs with improved functionality. Examples of such functionalities are the use of IBs for antigen or drug delivery and further equipping them with affinity binders (antibodies, Affibody® molecules, Nanobody® molecules) or carbohydrate/sugar molecules to target IBs to certain tissues or cell types. The coupling peptide and partner peptide are typically derived from a protein of a Gram positive or Gram negative bacterium, which protein is, and/or comprises a domain that is, capable of forming one or more isopeptide bonds.

A person of skill in the art will be familiar with proteins capable of forming one or more isopeptide bonds and non-limiting examples that may be suitable for use in the present invention include Spy0128 (Kang et al. 2007 Science 318(5856), 1625-28), Spy0125 (Pointon et al. 2010 J Biol Chem 285(44), 33858-66) and FbaB (Oke et al. 2010 J Struct Funct Genomics 11(2), 167-80) of Streptococcus pyogenes, fibronectin-binding protein CnaB of Streptococcus dysgalactiae (Pröschel et al. 2017 PLoS One 12(6)), Cna of Staphylococcus aureus (Kang et al. 2007 supra)), the ACE19 protein of Enterococcus faecalis (Kang et al. 2007 supra), the BcpA pilin of Bacillus cereus (Budzik et al. 2007 PNAS USA, 106(47), 19992-7), the minor pilin GBS52 of Streptococcus agalactiae (Kang et al. 2007 Science 318(5856), 1625-8), SpaA of Corynebacterium diphtheriae (Kang et al. 2009 PNAS USA 106(40), 16967-71), SpaP of Streptococcus mutans (Nylander et al. 2011 Acta Crystallogr Sect F Struct Biol Cryst Commun 67(Pt1), 23-6), RrgA (Izore et al. 2010 Structure 18(1), 106-15), RrgB and RrgC of Streptococcus pneumoniae (El Mortaji et al. 2010 J Biol Chem 285(16), 12405-15) and SspB of Streptococcus gordonii (Forsgren et al. 2010 J Mol Biol 397(3), 740-51).

Suitably, said protein is of a Gram positive bacterium of the Streptococcaceae family, such as Streptococcus pyogenes, Streptococcus pneumoniae or Streptococcus dysgalactiae. Thus, said protein may be adhesin RrgA of Streptococcus pneumoniae, fibronectin-binding protein FbaB of Streptococcus pyogenes, major pilin protein Spy0128 of Streptococcus pyogenes, or fibronectin-binding protein CnaB of Streptococcus dysgalactiae, or a protein with at least 70% sequence identity thereto which is capable of forming one or more isopeptide bonds.

In one embodiment, the inventors have made use of coupling peptides, such as peptide tags capable of forming spontaneous amide bonds based on harnessing reactions of adhesion proteins from the bacterium Streptococcus pyogenes (e.g. Spy0128 or FbaB), to link heterologous proteins to IBs. These include the irreversible peptide-protein interaction of the coupling peptide SpyTag with its affiliated partner peptide SpyCatcher. The IB is thus linked to the ligation system, the final construct being IB-SpyTag-SpyCatcher.

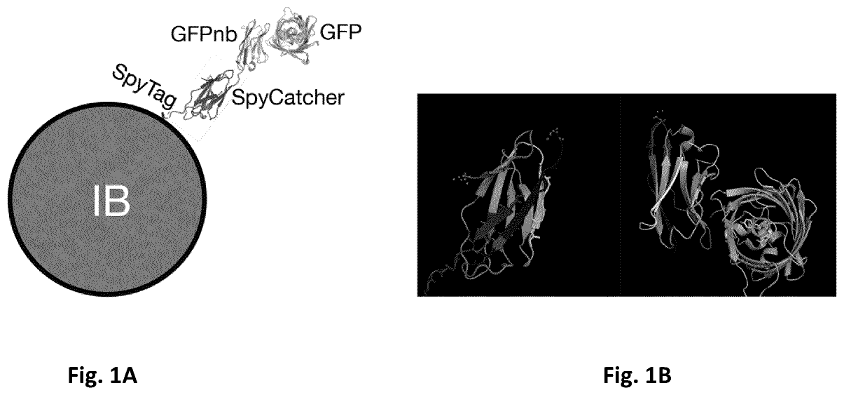

Example 1 and FIGS. 1A and 1B describes and illustrates the inventors' proof of concept for the decoration of IBs with functional affinity moieties to permit targeting of the IBs to specific targets, cells and/or tissues. In this particular example, the inclusion body (IB) was decorated with a green fluorescent protein Nanobody® molecule (GFPnb) with affinity for green fluorescent protein (GFP) using the ligation system of SpyTag and SpyCatcher (Zakeri et al. 2012 supra).

In another embodiment, a second ligation system is used, based on the adhesin RrgA from Streptococcus pneumoniae. This system comprises the coupling peptide (peptide tag) SnoopTag, which forms a spontaneous isopeptide bond to its partner peptide (binding protein partner) SnoopCatcher. In this embodiment, the inclusion body is linked to the second ligation system of SnoopTag and SnoopCatcher (Veggiani et al. 2016 supra), the final construct being IB-SnoopTag-SnoopCatcher.

Additional non-limiting examples of ligation systems that may be used to practise the present invention in a similar manner include SdyTag-SdyCatcher (Pröschel et al. 2017 PLoS One 12(6); Tan et al. PLoS One. 2016. 11(10)), SpyTag002-SpyCatcher002 (Keeble et al. 2017 Angew Chem Int Ed Engl December 22 56(52): 16521-16525), SpyTag003-SpyCatcher003 (Keeble et al. 2019 Proc Natl Acad Sci USA December 26 116(52): 26523-26533), SpyTag0128-SpyCatcher0128 (Zakeri & Howarth 2010 J Am Chem Soc 132(13); WO 2011/098772), KTag-SpyTag, SpyTag-KTag, DogTag-SnoopTagJr and SnoopTagJnr-DogTag (Buldun et al. 2018 J Am Chem Soc. February 28; 140(8):3008-3018), SpyTag-SpyCatcher002, SpyTag002-SpyCatcher, SpyTag-BDTag and BDTag-SpyTag (Wu et al. 2018 J Am Chem Soc November 19 140(50): 17474-17483).

In relation to the above ligation systems, the inventors have surprisingly found that the peptide KTag, which originates from the protein of SpyCatcher, and SpyTag may alternate between acting as a coupling peptide and a partner peptide to form irreversible peptide-protein interactions with both coupling peptides and partner peptides under the facilitation of SpyLigase. This means that when acting as a coupling peptide, KTag may bind to SpyTag, which acts as a partner peptide, to form a ligation system according to KTag-SpyTag. Therefore, in one embodiment of the disclosed inclusion body, the final construct may be IB-KTag-SpyTag. In another embodiment of the disclosed inclusion body, KTag may instead act as a partner peptide and bind to SpyTag, which in this instance acts as a coupling peptide, such that the final construct may be IB-SpyTag-KTag.

Similarly, in a tripartite system derived from adhesin RrgA of the bacterium Streptococcus pneumoniae, wherein the coupling peptide is DogTag and the partner peptide is SnoopTagJr, or wherein the coupling peptide is SnoopTagJr and the partner peptide is DogTag, the formation of the covalent isopeptide bond may be mediated by addition of SnoopLigase.

Previously being regarded as unwanted byproducts of protein production, proteins produced in inclusion bodies are today seen as functional nanoparticles with numerous potential applications in areas such as diagnostics, tissue engineering, drug delivery and antigen delivery. Hence, the inclusion body of the present invention typically comprises at least one protein of interest (POI) or a portion thereof.

In one embodiment, said protein of interest is a protein with a therapeutic purpose that treats a condition or disorder. Suitably, said condition or disorder is selected from the group consisting of cancer, autoimmune disease, inflammatory disease, transplant rejection and infectious disease.

In another embodiment, said protein of interest is a protein with a prophylactic purpose that protects against a condition or disorder. Suitably, said condition or disorder is selected from the group consisting of cancer, autoimmune disease, inflammatory disease, transplant rejection and infectious disease.

Preferably, said protein of interest is an antigen or a fragment thereof. Said antigen may be selected from the group consisting of an antigen from an infectious organism, a tumor antigen, a tumor stroma antigen and a tumor associated antigen.

The characteristics of the POI expressed in or as an IB may vary. It may for example be a soluble or partly soluble cytoplasmic polypeptide, a soluble or partly soluble secretory polypeptide or a membrane polypeptide.

The function of the POI may also vary. It may for example constitute a bioactive molecule, such as an antigen for immunization, a therapeutic or curative agent against disease (e.g. a growth factor, hormone, interleukin, interferon or other polypeptide that affects cellular components such as receptors, channels and lipids), an enzyme, a toxin, a structural polypeptide, a research tool, such as green fluorescent protein (GFP), or an antimicrobial polypeptide.

Operably fusing an IBFS to a POI sequence forms a fusion polypeptide. The sequences are easily fused together in, for example, a vector using well-known recombinant DNA techniques. Said fusion polypeptide may comprise more than one POI. Thus, the fusion polypeptide may comprise two, three or more POIs. The possibly several POIs of the fusion polypeptide may have different characteristics and functions. Whenever referred to herein in singular form, the POI may also alternatively be present as two, three or more POIs.

The components of fusion polypeptides are fused in such a way that they form one continuous polypeptide. The one or several POI may be adjacent to the inclusion body forming sequence. Alternatively, the fusion polypeptide may comprise an intermediate amino acid sequence between the POI and the inclusion body forming sequence. Moreover, there is no limitation in the order of the POI relative to the inclusion body forming sequence. Because design of the fusion polypeptide is carried out at the DNA level, care must be taken so that the reading frame of the POI is the same as the reading frame of the inclusion body forming sequence.

Inclusion bodies form naturally in some environments. Also, when there is a need to specifically express peptides of interest (POI) in inclusion bodies, an inclusion body forming sequence (IBFS), for example the signal sequence ssTorA, may be used. Through techniques well-known to the skilled person, such an IBFS is genetically fused to a sequence expressing the POI and, as a result of such a fusion, the POI is expressed in insoluble inclusion bodies (IBs). An IBFS may be fused to a POI of any length, function and solubility for production of the POI in inclusion bodies. Normally, the production of a POI is increased when expressed and aggregated in an insoluble inclusion body due to being protected from proteolytic degradation. Further on, the host cell is protected from any toxicity of the POI. Inclusion bodies comprising the POI are easy to separate from other proteins and cellular components e.g. by centrifugation and/or filtration.

A person of skill in the art will be familiar with IBFS other than ssTorA, including TrpΔLE, ketosteroid isomerase, β-galactosidase, PagP, EDDIE, ELK16, GFIL8, PaP3.30, TAF12-HFD and the F4 fragment of PurF. Other IBFS:s suitable for use in the present invention are the sequences based on a minimal motif from ssTorA described in WO2018/138316.

The POI may optionally also comprise additional portions of amino acid sequence for other functions, e.g. amino acid tags for use in purification or biochemical detection of the POI, such as a hexa-His tag.

In one embodiment, the POI may be an antigen which sequence can be linked to an IBFS and/or a coupling peptide (e.g. a peptide tag) sequence resulting in a fusion polypeptide. Said fusion polypeptide may comprise more than one POI being an antigen but may also comprise other kinds of adjacently linked POIs.

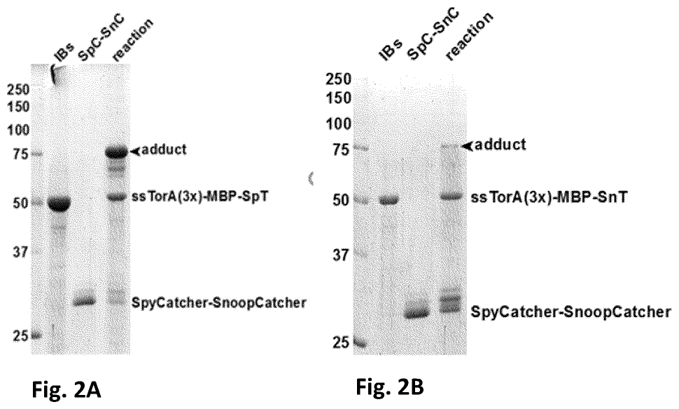

In Example 2, the inventors verified their findings by successfully conjugating the two different coupling peptides (peptide tags) SpyTag and SnoopTag to their cognate SpyCatcher and SnoopCatcher, respectively. Inclusion bodies were produced using the fusion protein ssTorA(3×)-MBP-coupling peptide, wherein ssTorA(3×) is three copies of the IBFS ssTorA genetically fused to maltose binding protein (MBP), which is known to successfully produce inclusion bodies with ssTorA(3×), and a C-terminal coupling peptide (peptide tag), either a SpyTag (ssTorA(3×)-MBP-SpT) or SnoopTag (ssTorA(3×)-MBP-SnT). Each fusion protein was then incubated with soluble SpyCatcher-SnoopCatcher a fusion protein comprising an N-terminal SpyCatcher moiety and a C-terminal SnoopCatcher moiety. SDS-page analysis shows that an adduct is formed using either of the systems according to ssTorA(3×)-MBP-SpT/SnT-SpyCatcher-SnoopCatcher (FIG. 2A and FIG. 2B). The coupling peptide (peptide tag) is typically C-terminal in the POI but may also be N-terminal or internal. By “internal” is meant that the peptide tag is located at least 1, at least 2, at least 5, at least 10, at least 15, at least 20, at least 25, or at least 30 amino acids in from the N-terminal and C-terminal ends (also referred to herein as the N-terminus and C-terminus, respectively) of the POI.

Fusing an IBFS to the coupling peptide sequence and the POI sequence for the formation of inclusion bodies is possible but not necessary in the current disclosure. However, embodiments where IBFS have been used also form fusion polypeptides according to IBFS-IB-coupling peptide and are subject to the same conditions as fusion polypeptides where IBFS have not been used in terms of, for example, the characteristics of the POI.

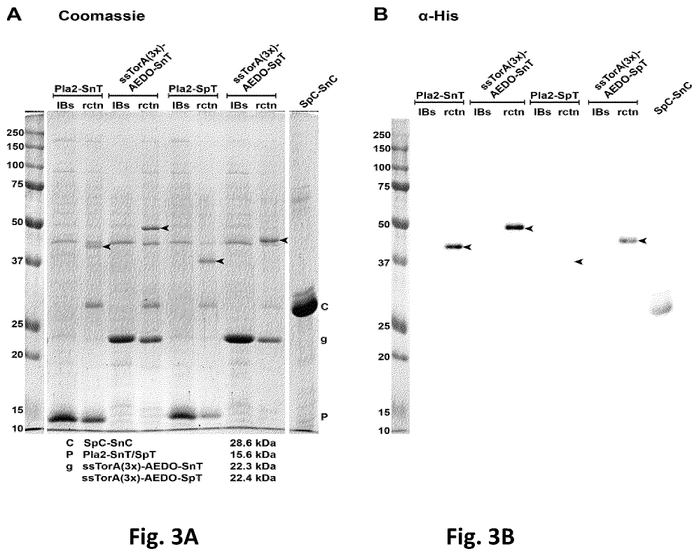

In view of the foregoing, it will be appreciated that an inclusion body may be produced with or without an IBFS. The inventors have, for example, managed to decorate spontaneously formed IBs by using human recombinant protein phospholipase 2 (Pla2) which carries a coupling peptide such as SpyTag or SnoopTag on its surface and may be linked to a SpyCatcher, SnoopCatcher or KTag partner peptide. Example 3 more elaborately discloses how the inventors have successfully decorated both ssTorA(3×)-induced and ssTorA(3×)-independent inclusion bodies with SpyTag or SnoopTag. The inventors have used human recombinant protein (Pla2) expressed in the form of inclusion bodies carrying either a C-terminal SpyTag or SnoopTag and covalently coupled the tags to a SpyCatcher-SnoopCatcher (SpC-SnC) fusion protein. SDS-PAGE analysis (FIG. 3A) verifies the coupling by showing bands representing the Pla2-SpT-SpC-SnC conjugation adduct. A polyHistidine detection tag incorporated in the SpyCatcher-SnoopCatcher fusion protein enabled further verification by Western blotting using antibody recognition (FIG. 3B). Using similar methodology, coupling of SpyCatcher-SnoopCatcher to IBs formed by a polypeptide comprising an N-terminal IBFS (ssTorA[3×]), a C-terminal SpyTag or SnoopTag, and short antigenic epitopes of different origin in between (ssTorA(3×)-AEDO-SpT and ssTorA(3×)-AEDO-SnT) is demonstrated.

When using an IBFS, the inventors produced inclusion bodies using the fusion protein ssTorA(3×)-MBP-SnT or ssTorA(3×)-MBP-SpT: triple TorA signal sequence for inclusion body formation, maltose binding protein (MBP) which is known to produce good inclusion bodies with ssTorA(3×), and a C-terminal SnoopTag or SpyTag respectively. The skilled person realizes that ssTorA, MBP and AEDO are merely examples of IBFS, proteins and antigens that may be used in the formation of inclusion bodies and that the current disclosure is in no way limited to the use of these examples. This should be kept in mind when studying Table 1 below, which presents an overview of successful protein ligation to inclusion bodies, wherein MBP, AEDO and ssTorA serve as illustrative examples of how to make use of the inclusion body of the current invention.

TABLE 1
Overview of successful protein ligation to inclusion bodies

| IB protein | Coupled protein |
|---|---|
| (incl. coupling peptide) | (incl. partner peptide) |
| Pla2-SnoopTag | SpyCatcher-SnoopCatcher |
| Pla2-SpyTag | SpyCatcher-SnoopCatcher |
| ssTorA(3x)-AEDO-SnoopTag | SpyCatcher-SnoopCatcher |
| ssTorA(3x)-AEDO-SpyTag | SpyCatcher-SnoopCatcher |
| ssTorA(3x)-MBP-SpyTag | SpyCatcher-SnoopCatcher |
| ssTorA(3x)-MBP-SnoopTag | SpyCatcher-SnoopCatcher |
| ssTorA(3x)-MBP-SpyTag | GFPnb-SpyCatcher (+ GFP) |
| ssTorA(3x)-MBP-KTag | SnoopTag-mEGFP-SpyTag + SpyLigase |
| ssTorA(3x)-AEDO-SpyTag | SpyCatcher002-ZZ |
| ssTorA(3x)-AEDO-SpyTag | SpyCatcher002-Protein A/G |

Additional examples of IBs (including coupling peptide) and partner peptides that may be suitable are listed in Table 2 below.

TABLE 2
Additional examples of IBs (incl. coupling
peptide) and partner peptides

| IB | Coupled protein (incl. | |
|---|---|---|
| protein | partner peptide) | Remark |
| ssTorA(3X)-MBP- | SpyCatcher- | Coupled protein |
| SpyTag | SnoopCatcher | is presented in |
| | | bacterial lysate |
| ssTorA(3X)-MBP- | ZZ- | Coupled protein is |
| SpyTag | SpyCatcher | presented in |
| | | bacterial lysate and |
| | | antibodies are |
| | | bound to conjugated IBs |
| ssTorA(3X)-MBP- | Protein A/G- | Coupled protein is |
| SpyTag | SpyCatcher | presented in |
| | | bacterial lysate |
| | | and antibodies are |
| | | bound to conjugated IBs |
| ssTorA(3X)-MBP- | GFP nanobody- | Coupled protein is |
| SpyTag | SpyCatcher | presented in |
| | | bacterial lysate |
| | | and antibodies are |
| | | bound to conjugated IBs |
| ssTorA(3X)-TrxA- | SpyCatcher- | |
| SpyTag | SnoopCatcher | |
| ssTorA(3X)-TrxA- | ZZ-SpyCatcher | +antibodies are bound to |
| SpyTag | | conjugated IBs |
| ssTorA(3X)-TrxA- | Protein A/G- | +antibodies are bound to |
| SpyTag | SpyCatcher | conjugated IBs |
| ssTorA(3X)-TrxA- | GFP nanobody- | +GFP is bound |
| SpyTag | SpyCatcher | to conjugated IBs |
| ssTorA(3X)-TrxA- | SpyCatcher- | Coupled protein is |
| SpyTag | SnoopCatcher | presented in |
| | | bacterial lysate |
| ssTorA(3X)-TrxA- | ZZ-SpyCatcher | Coupled protein is |
| SpyTag | | presented in |
| | | bacterial lysate and |
| | | antibodies are |
| | | bound to conjugated IBs |
| ssTorA(3X)-TrxA- | Protein A/G- | Coupled protein is |
| SpyTag | SpyCatcher | presented in |
| | | bacterial lysate |
| | | and antibodies are |
| | | bound to conjugated IBs |
| ssTorA(3X)-TrxA- | GFP nanobody- | Coupled protein is |
| SpyTag | SpyCatcher | presented in |
| | | bacterial lysate |
| | | and GFP is bound |
| | | to conjugated IBs |
| ssTorA(3X)-antigen- | SpyCatcher- | |
| SpyTag | SnoopCatcher | |
| ssTorA(3X)-antigen- | ZZ-SpyCatcher | +antibodies are bound to |
| SpyTag | | conjugated IBs |
| ssTorA(3X)-antigen- | Protein A/G- | +antibodies are bound to |
| SpyTag | SpyCatcher | conjugated IBs |
| ssTorA(3X)-antigen- | GFP nanobody- | +and GFP is bound to |
| SpyTag | SpyCatcher | conjugated IBs |
| ssTorA(3X)-MBP- | SpyCatcher- | |
| SnoopTag | SnoopCatcher | |
| ssTorA(3X)-MBP- | ZZ-SnoopCatcher | +antibodies are bound to |
| SnoopTag | | conjugated IBs |
| ssTorA(3X)-MBP- | Protein A/G- | +antibodies are bound to |
| SnoopTag | SnoopCatcher | conjugated IBs |
| ssTorA(3X)-MBP- | GFP nanobody- | +GFP is bound to |
| SnoopTag | SnoopCatcher | conjugated IBs |
| ssTorA(3X)-TrxA- | SpyCatcher- | |
| SnoopTag | SnoopCatcher | |
| ssTorA(3X)-TrxA- | ZZ-SpyCatcher | +antibodies are bound to |
| SnoopTag | | conjugated IBs |
| ssTorA(3X)-TrxA- | Protein A/G- | +antibodies are bound to |
| SnoopTag | SnoopCatcher | conjugated IBs |
| ssTorA(3X)-TrxA- | GFP nanobody- | +GFP is bound to |
| SnoopTag | SnoopCatcher | conjugated IBs |
| ssTorA(3X)-antigen- | SpyCatcher- | |
| SnoopTag | SnoopCatcher | |
| ssTorA(3X)-antigen- | ZZ-SpyCatcher | +antibodies are bound to |
| SnoopTag | | conjugated IBs |
| ssTorA(3X)-antigen- | Protein A/G- | +antibodies are bound to |
| SnoopTag | SnoopCatcher | conjugated IBs |
| ssTorA(3X)-antigen- | GFP nanobody- | +GFP is bound to |
| SnoopTag | SnoopCatcher | conjugated IBs |
| ssTorA(3X)-MBP- | KTag-model | Coupling is catalyzed |
| SpyTag | protein | by added SpyLigase |
| ssTorA(3X)-TrxA- | KTag-model | Coupling is catalyzed |
| SpyTag | protein | by added SpyLigase |
| ssTorA(3X)-antigen- | KTag-model | Coupling is catalyzed |
| SpyTag | protein | by added SpyLigase |
| ssTorA(3X)-MBP- | SpyCatcher002-model | Coupling is catalyzed |
| SpyTag002 | protein | by added SpyLigase |
| ssTorA(3X)-TrxA- | SpyCatcher002-model | Coupling is catalyzed |
| SpyTag002 | protein | by added SpyLigase |
| ssTorA(3X)-antigen- | SpyCatcher002-model | Coupling is catalyzed |
| SpyTag002 | protein | by added SpyLigase |
| ssTorA(3X)-MBP- | SdyCatcher-model | |
| SdyTag | protein | |
| ssTorA(3X)-TrxA- | SdyCatcher-model | |
| SdyTag | protein | |
| ssTorA(3X)-antigen- | SdyCatcher-model | |
| SdyTag | protein | |
| ssTorA(3X)-MBP- | SnoopCatcher-model | |
| SnoopTag | protein | |
| ssTorA(3X)-TrxA- | SnoopCatcher-model | |
| SnoopTag | protein | |
| ssTorA(3X)-antigen- | SnoopCatcher-model | |
| SnoopTag | protein | |
| ssTorA(3X)-MBP- | DogTag-model | Coupling is catalyzed |
| SnoopTagJnr | protein | by added SnoopLigase |
| ssTorA(3X)-TrxA- | DogTag-model | Coupling is catalyzed |
| SnoopTagJnr | protein | by added SnoopLigase |
| ssTorA(3X)-antigen- | DogTag-model | Coupling is catalyzed |
| SnoopTagJnr | protein | by added SnoopLigase |
| ssTorA(3X)-MBP- | SnoopTagJnr-model | Coupling is catalyzed |
| DogTag | protein | by added SnoopLigase |
| ssTorA(3X)-TrxA- | SnoopTagJnr- | Coupling is catalyzed |
| DogTag | model | by added SnoopLigase |
| | protein | |
| ssTorA(3X)-antigen- | SnoopTagJnr- | Coupling is catalyzed |
| DogTag | model | by added SnoopLigase |
| | protein | |
| ssTorA(3X)-MBP- | SpyCatcher- | |
| SpyTag | antibody | |
| ssTorA(3X)-TrxA- | SpyCatcher- | |
| SpyTag | antibody | |
| ssTorA(3X)-antigen- | SpyCatcher- | |
| SpyTag | antibody | |
| ssTorA(3X)-MBP- | SpyCatcher002- | |
| SpyTag002 | antibody | |
| ssTorA(3X)-TrxA- | SpyCatcher002- | |
| SpyTag002 | antibody | |
| ssTorA(3X)-antigen- | SpyCatcher002- | |
| SpyTag002 | antibody | |
| ssTorA(3X)-MBP- | SnoopCatcher- | |
| SnoopTag | antibody | |
| ssTorA(3X)-TrxA- | SnoopCatcher- | |
| SnoopTag | antibody | |
| ssTorA(3X)-antigen- | SnoopCatcher- | |
| SnoopTag | antibody | |
| ssTorA(3X)-MBP- | SpyTag- | |
| SpyCatcher | antibody | |
| ssTorA(3X)-TrxA- | SpyTag- | |
| SpyCatcher | antibody | |
| ssTorA(3X)-antigen- | SpyTag- | |
| SpyCatcher | antibody | |
| ssTorA(3X)-MBP- | SnoopTag- | |
| SnoopCatcher | antibody | |
| ssTorA(3X)-TrxA- | SnoopTag- | |
| SnoopCatcher | antibody | |
| ssTorA(3X)-antigen- | SnoopTag- | |
| SnoopCatcher | antibody | |
| ssTorA(3X)-MBP- | SpyTag- | Coupling is catalyzed |
| KTag | antibody | by added SpyLigase |
| ssTorA(3X)-TrxA- | SpyTag- | Coupling is catalyzed |
| KTag | antibody | by added SpyLigase |
| ssTorA(3X)-antigen- | SpyTag- | Coupling is catalyzed |
| KTag | antibody | by added SpyLigase |
| ssTorA(3X)-MBP- | DogTag- | Coupling is catalyzed |
| SnoopTagJnr | antibody | by added SnoopLigase |
| ssTorA(3X)-TrxA- | DogTag- | Coupling is catalyzed |
| SnoopTagJnr | antibody | by added SnoopLigase |
| ssTorA(3X)-antigen- | DogTag- | Coupling is catalyzed |
| SnoopTagJnr | antibody | by added SnoopLigase |
| ssTorA(3X)-MBP- | SpyCatcher- | |
| SpyTag | nanobody | |
| ssTorA(3X)-TrxA- | SpyCatcher- | |
| SpyTag | nanobody | |
| ssTorA(3X)-antigen- | SpyCatcher- | |
| SpyTag | nanobody | |
| ssTorA(3X)-MBP- | SpyCatcher002- | |
| SpyTag002 | nanobody | |
| ssTorA(3X)-TrxA- | SpyCatcher002- | |
| SpyTag002 | nanobody | |
| ssTorA(3X)-antigen- | SpyCatcher002- | |
| SpyTag002 | nanobody | |
| ssTorA(3X)-MBP- | SnoopCatcher- | |
| SnoopTag | nanobody | |
| ssTorA(3X)-TrxA- | SnoopCatcher- | |
| SnoopTag | nanobody | |
| ssTorA(3X)-antigen- | SnoopCatcher- | |
| SnoopTag | nanobody | |
| ssTorA(3X)-MBP- | SpyTag- | |
| SpyCatcher | nanobody | |
| ssTorA(3X)-TrxA- | SpyTag- | |
| SpyCatcher | nanobody | |
| ssTorA(3X)-antigen- | SpyTag- | |
| SpyCatcher | nanobody | |
| ssTorA(3X)-MBP- | SnoopTag- | |
| SnoopCatcher | nanobody | |
| ssTorA(3X)-TrxA- | SnoopTag- | |
| SnoopCatcher | nanobody | |
| ssTorA(3X)-antigen- | SnoopTag- | |
| SnoopCatcher | nanobody | |
| ssTorA(3X)-MBP- | SpyTag- | Coupling is catalyzed |
| KTag | nanobody | by added SpyLigase |
| ssTorA(3X)-TrxA- | SpyTag- | Coupling is catalyzed |
| KTag | nanobody | by added SpyLigase |
| ssTorA(3X)-antigen- | SpyTag- | Coupling is catalyzed |
| KTag | nanobody | by added SpyLigase |
| ssTorA(3X)-MBP- | DogTag- | Coupling is catalyzed |
| SnoopTagJnr | nanobody | by added SnoopLigase |
| ssTorA(3X)-TrxA- | DogTag- | Coupling is catalyzed |
| SnoopTagJnr | nanobody | by added SnoopLigase |
| ssTorA(3X)-antigen- | DogTag- | Coupling is catalyzed |
| SnoopTagJnr | nanobody | by added SnoopLigase |
| KSI-MBP-SpyTag | SnoopCatcher- | |
| | model | |
| | protein | |
| KSI-TrxA-SpyTag | SnoopCatcher- | |
| | model | |
| | protein | |
| KSI-antigen-SpyTag | SnoopCatcher- | |
| | model | |
| | protein | |
| KSI-MBP-SnoopTag | SnoopCatcher- | |
| | model | |
| | protein | |
| KSI-TrxA-SnoopTag | SnoopCatcher- | |
| | model | |
| | protein | |
| KSI-antigen- | SnoopCatcher- | |
| SnoopTag | model | |
| | protein | |
| ssTorA(3X)-MBP- | SpyCatcher- | |
| SpyTag | glycan | |
| ssTorA(3X)-TrxA- | SpyCatcher- | |
| SpyTag | glycan | |
| ssTorA(3X)-antigen- | SpyCatcher- | |
| SpyTag | glycan | |
| ssTorA(3X)-MBP- | SpyCatcher- | |
| SpyTag | adjuvant | |
| ssTorA(3X)-TrxA- | SpyCatcher- | |
| SpyTag | adjuvant | |
| ssTorA(3X)-antigen- | SpyCatcher- | |
| SpyTag | adjuvant | |
| ssTorA(3X)-MBP- | SpyCatcher- | |
| SpyTag | cytokine | |
| ssTorA(3X)-TrxA- | SpyCatcher- | |
| SpyTag | cytokine | |
| ssTorA(3X)-antigen- | SpyCatcher- | |
| SpyTag | cytokine | |
| ssTorA(3X)-MBP- | SpyCatcher- | |
| SpyTag | adhesin | |
| ssTorA(3X)-TrxA- | SpyCatcher- | |
| SpyTag | adhesin | |
| ssTorA(3X)-antigen- | SpyCatcher- | |
| SpyTag | adhesin | |
| ssTorA(3X)-MBP- | KTag- | Coupling is catalyzed |
| SpyTag | glycan | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | KTag- | Coupling is catalyzed |
| SpyTag | glycan | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | KTag- | Coupling is catalyzed |
| SpyTag | glycan | by added |
| | | SpyLigase/SpyTag and |
| | | KTag may |
| | | be replaced by components |
| | | SnoopTagJnr/DogTag/ |
| | | SnoopLigase system |
| ssTorA(3X)-MBP- | KTag- | Coupling is catalyzed |
| SpyTag | adjuvant | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | KTag- | Coupling is catalyzed |
| SpyTag | adjuvant | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | KTag- | Coupling is catalyzed |
| SpyTag | adjuvant | by added |
| | | SpyLigase/SpyTag and |
| | | KTag may |
| | | be replaced by components |
| | | SnoopTagJnr/DogTag/ |
| | | SnoopLigase system |
| ssTorA(3X)-MBP- | KTag- | Coupling is catalyzed |
| SpyTag | cytokine | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | KTag- | Coupling is catalyzed |
| SpyTag | cytokine | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | KTag- | Coupling is catalyzed |
| SpyTag | cytokine | by added |
| | | SpyLigase/SpyTag and |
| | | KTag may |
| | | be replaced by components |
| | | SnoopTagJnr/DogTag/ |
| | | SnoopLigase system |
| ssTorA(3X)-MBP- | KTag- | Coupling is catalyzed |
| SpyTag | adhesin | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | KTag- | Coupling is catalyzed |
| SpyTag | adhesin | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | KTag- | Coupling is catalyzed |
| SpyTag | adhesin | by added |
| | | SpyLigase/SpyTag and |
| | | KTag may |
| | | be replaced by components |
| | | SnoopTagJnr/DogTag/ |
| | | SnoopLigase system |
| ssTorA(3X)-MBP- | SpyTag- | Coupling is catalyzed |
| KTag | glycan | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | SpyTag- | Coupling is catalyzed |
| KTag | glycan | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | SpyTag- | Coupling is catalyzed |
| KTag | glycan | by added |
| | | SpyLigase/SpyTag and |
| | | KTag may |
| | | be replaced by components |
| | | SnoopTagJnr/DogTag/ |
| | | SnoopLigase system |
| ssTorA(3X)-MBP- | SpyTag- | Coupling is catalyzed |
| KTag | adjuvant | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | SpyTag- | Coupling is catalyzed |
| KTag | adjuvant | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | SpyTag- | Coupling is catalyzed |
| KTag | adjuvant | by added |
| | | SpyLigase/SpyTag and |
| | | KTag may |
| | | be replaced by components |
| | | SnoopTagJnr/DogTag/ |
| | | SnoopLigase system |
| ssTorA(3X)-MBP- | SpyTag- | Coupling is catalyzed |
| KTag | cytokine | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | SpyTag- | Coupling is catalyzed |
| KTag | cytokine | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | SpyTag- | Coupling is catalyzed |
| KTag | cytokine | by added |
| | | SpyLigase/SpyTag and |
| | | KTag may |
| | | be replaced by components |
| | | SnoopTagJnr/DogTag/ |
| | | SnoopLigase system |
| ssTorA(3X)-MBP- | SpyTag- | Coupling is catalyzed |
| KTag | adhesin | by added |
| | | SpyLigase/SpyTag and |
| | | KTag may |
| | | be replaced by components |
| | | SnoopTagJnr/DogTag/ |
| | | SnoopLigase system |
| ssTorA(3X)-TrxA- | SpyTag- | Coupling is catalyzed |
| KTag | adhesin | by added |
| | | SpyLigase/SpyTag and |
| | | KTag may |
| | | be replaced by components |
| | | SnoopTagJnr/DogTag/ |
| | | SnoopLigase system |
| ssTorA(3X)-antigen- | SpyTag- | Coupling is catalyzed |
| KTag | adhesin | by added |
| | | SpyLigase/SpyTag and |
| | | KTag may |
| | | be replaced by components |
| | | SnoopTagJnr/DogTag/ |
| | | SnoopLigase system |

As described previously, the present disclosure demonstrates a powerful method for decorating inclusion bodies with a variety of additional moieties using a variety of different ligation systems. Suitably, the additional moieties are attached, linked or otherwise coupled to the partner peptide.

The additional moiety may constitute any bioactive molecule, such as a curative agent against disease (e.g. a growth factor, hormone, interleukin, interferon or other polypeptide that affects cellular components such as receptors, channels and lipids), an enzyme, a toxin, a structural polypeptide, a research tool, such as green fluorescent protein (GFP), or an antimicrobial polypeptide.

Non-limiting examples of additional moieties include immune modulating or targeting compounds such as, for example, a cytokine, adjuvant, antibody, Affibody® molecule, Nanobody® molecule, DARPIN, PAMPs, TLR ligands or agonists, lipids, RNA, DNA, immunomodulating peptides, peptidomimetics, T helper cell epitopes, immune checkpoint inhibitor, PLGA, chitosan, TRAIL, IL-1, IL-2, IL-3, IL-4, IL-5, IL-7, IL-8, IL-9, IL-10R DN or a subunit thereof, IL-15, IL-18, IL-21, IL-23, IL-24, IL-27, GM-CSF, IFN-alpha, IFN-gamma, CCL3 (MIP-Ia), CCL5 (RANTES), CCL7 (MCP3), XCLI (lymphotactin), CXCLI (MGSA-alpha), CCR7, CCL 19 (MIP-3b), CXCL9 (MIG), CXCL10 (IP-10), CXCL 12 (SDF-I), CCL21 (6Ckine), OX40L, 4-IBBL, CD40, CD70, GITRL, LIGHT, b-Defensin, HMGBI, Flt3L, IFN-beta, TNF, dnFADD, TGF-alpha, PD-LIRNAi, a PD-L1 antisense oligonucleotide, TGFbRII DN, ICOS-L and SIOO. In some embodiments, the additional moiety of the inclusion body is a moiety that directs IBs to specific subcellular locations, cells or tissues, such as a glycan, adjuvant, adhesin, enzyme, traceable probe, antibody, Affibody® molecule, Nanobody® molecule, DARPIN, toxin, drug, chemotherapeutic drug, components of the complement system, hormone. This enables the use of inclusion bodies for targeting, making them suitable for antigen or drug delivery.

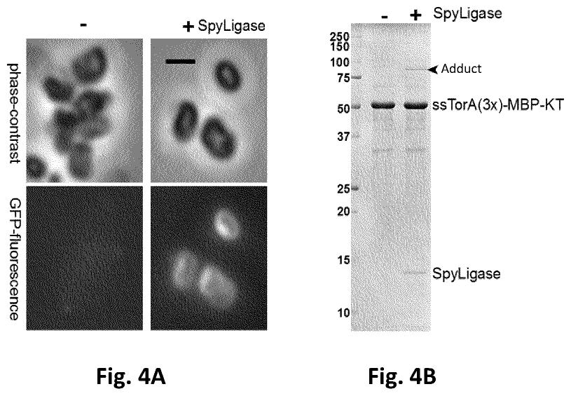

Thus, after a first decoration of the disclosed inclusion body using a ligation system, the inventors have further shown that it is possible for said inclusion body to undergo a second decoration by linking the partner peptide to an additional moiety. In Example 4 of this disclosure, the additional moiety is a soluble mEGFP (monomeric enhanced green fluorescent protein) derivative carrying a SnoopTag at its N-terminus and a SpyTag at its C-terminus (SnT-mEGFP-SpT). This was mixed with ssTorA(3×)-MBP IBs carrying a genetically fused KTag (ssTorA(3×)-MBP-KT). As previously mentioned, SpyLigase is needed for the bond between the K-Tag and the SpyTag to form, and was therefore added to one batch while leaving one batch without. Fluorescence microscopy analysis (FIG. 4A) verified the need for SpyLigase to drive the bond formation, as no signals were emitted from the batch without SpyLigase but clear signals were emitted from the batch with SpyLigase. The samples were subsequently analyzed using SDS-PAGE, which further verified the findings as a band was seen symbolizing the adduct at ˜100 kDa (FIG. 4B).

Consequently, by successfully expressing the coupling peptide as part of the IB (e.g. displayed on its surface) and maintaining its binding specificity towards the partner peptide, the inventors have successfully linked the IB to a specific compound, e.g. a functional moiety. The skilled person will appreciate that this unlocks a variety of possible application areas, such as using the disclosed decorated inclusion body as an immunostimulatory tool.

By varying the additional moiety, the decorated inclusion body constitutes a biomedical tool with great potential. The inventors have shown that it is possible to link a POI expressed in the form of an IB to a specific target. In a case where, for example, the POI is an antigen and the additional moiety has affinity for a specific cell of the immune system, the inclusion body provides a system for targeted antigen delivery. Thus, in one embodiment, the additional moiety is a targeting moiety that has affinity for a surface exposed component, such as a receptor.

Non-limiting examples of surface exposed components for which the targeting moiety may have an affinity are CD4, CD8, CD1, CD180, IgA, IgD, IgE, IgG, IgM, TCR, CRDs, Toll-like receptors (TLRs), nucleotide-binding oligomerization domain-like receptors (NLRs), retinoic acid-inducible gene I-like helicases receptors (RLRs), and C-type lectin receptors (CLRs), endocytic receptors, CD205/DEC205, CD209/DC-SIGN, Clec9A/DNGR-1/CD370, Clec7A/Dectin-1/CD369, Clec6A/Dectin-2, Clec12A, CD1d, CD11c, CD11 b, CD40, CD152/CTLA-4, CD279/PD-1, NOD-like receptors, RIG-I-like receptors, PRRs, CCRs, CD36, Siglec H, PDCTREM, Langerin, MMR, D-SIGN and Folate receptors.

In another embodiment, when the POI is a protein with therapeutic properties, the disclosed, targeted delivery system may be suitable for directing a medicament produced in the form of an IB to certain areas of the body. Examples include, but are not limited to, directing the IB to damaged or diseased tissues and cells. In one embodiment of the disclosure, the targeting moiety has an affinity for at least one tumor cell of any type of cancer. The cancer may be selected from the group consisting of lymphoma, leukemia, myeloma, lung cancer, non-small cell lung cancer (NSCLC), melanoma, renal cell cancer, ovarian cancer, glioblastoma, Merkel cell carcinoma, bladder cancer, head and neck cancer, colorectal cancer, esophageal cancer, cervical cancer, gastric cancer, hepatocellular cancer, prostate cancer, breast cancer, pancreatic cancer, and thyroid cancer.

In another embodiment, the targeting moiety is an antibody. The skilled person will appreciate that this embodiment may be applicable in a vast number of areas, for example immunotherapy, immunization, vaccine delivery and immune activation. Thus, the targeting moiety may be an antibody that is suitable for directing a protein of interest with a prophylactic purpose to certain areas of the body. In one embodiment, the antibody is a monoclonal, polyclonal or domain antibody (e.g. a Nanobody® molecule or a dAb).

As is well known, antibodies are immunoglobulin molecules capable of specifically binding to a target (an antigen), such as a carbohydrate, polynucleotide, lipid, polypeptide or other, through at least one antigen recognition site located in the variable region of the immunoglobulin molecule. As used herein, the term “antibody or an antigen binding fragment thereof” encompasses not only full-length or intact polyclonal or monoclonal antibodies, but also antigen-binding fragments thereof, such as Fab, Fab′, F(ab′)₂, Fab₃, Fv and variants thereof, fusion proteins comprising one or more antibody portions, humanized antibodies, chimeric antibodies, minibodies, diabodies, triabodies, tetrabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies and covalently modified antibodies. Further examples of modified antibodies and antigen binding fragments thereof include Nanobody® molecules, AlbudAbs, DARTs (dual affinity re-targeting), BiTEs (bispecific T-cell engager), TandAbs (tandem diabodies), DAFs (dual acting Fab), two-in-one antibodies, SMIPs (small modular immunopharmaceuticals), FynomAbs (fynomers fused to antibodies), DVD-Igs (dual variable domain immunoglobulin), CovX-bodies (peptide modified antibodies), duobodies and triomAbs. This listing of variants of antibodies and antigen binding fragments thereof is not to be seen as limiting.

The skilled person is furthermore aware of suitable affinity molecules of non-antibody origin, including DARPINS, Affibody® molecules, staphylococcal Protein A, streptococcal Protein G, Protein NG chimera, Protein L, or the Protein A domain derivatives protein Z and the ZZ dimer thereof. It will be appreciated that these molecules bind specifically to immunoglobulins. Thus, decoration of IBs with Protein A/G/L or their derivatives through isopeptide bonding technology as described herein allows for these IBs to be subsequently equipped with off-the-shelf antibodies.

The term “full-length antibody” as used herein refers to an antibody of any class, such as IgD, IgE, IgG, IgA, IgM or IgY (or any sub-class thereof). The subunit structures and three-dimensional configurations of different classes of antibodies are well known.

An “antigen binding fragment” is a portion or region of an antibody molecule, or a derivative thereof, that retains all or a significant part of the antigen binding of the corresponding full-length antibody. An antigen binding fragment may comprise the heavy chain variable region (V_{H}), the light chain variable region (V_{L}), or both. Each of the V_{H} and V_{L} typically contains three complementarity determining regions CDR1, CDR2 and CDR3. The three CDRs in V_{H} or V_{L} are flanked by framework regions (FR1, FR2, FR3 and FR4). As briefly listed above, examples of antigen binding fragments include, but are not limited to: (1) a Fab fragment, which is a monovalent fragment having a V_{L}-CL chain and a V_{H}-C_{H}1 chain; (2) a Fab′ fragment, which is a Fab fragment with the heavy chain hinge region, (3) a F(ab′)₂ fragment, which is a dimer of Fab′ fragments joined by the heavy chain hinge region, for example linked by a disulfide bridge at the hinge region; (4) an Fc fragment; (5) an Fv fragment, which is the minimum antibody fragment having the V_{L} and V_{H} domains of a single arm of an antibody; (6) a single chain Fv (scFv) fragment, which is a single polypeptide chain in which the V_{H} and V_{L} domains of an scFv are linked by a peptide linker; (7) an (scFv)₂, which comprises two V_{H} domains and two V_{L} domains, which are associated through the two V_{H} domains via disulfide bridges and (8) domain antibodies, which can be antibody single variable domain (V_{H} or V_{L}) polypeptides that specifically bind antigens.

Antigen binding fragments can be prepared via routine methods. For example, F(ab′)₂ fragments can be produced by pepsin digestion of a full-length antibody molecule, and Fab fragments can be generated by reducing the disulfide bridges of F(ab′)₂ fragments. Alternatively, fragments can be prepared via recombinant technology by expressing the heavy and light chain fragments in suitable host cells (e.g., E. coli, yeast, mammalian, plant or insect cells) and having them assembled to form the desired antigen-binding fragments either in vivo or in vitro. A single-chain antibody can be prepared via recombinant technology by linking a nucleotide sequence coding for a heavy chain variable region and a nucleotide sequence coding for a light chain variable region. For example, a flexible linker may be incorporated between the two variable regions. The skilled person is aware of methods for the preparation of both full-length antibodies and antigen binding fragments thereof.

Thus, in one embodiment, this aspect of the disclosure provides an inclusion body comprising a targeting moiety that is an antibody or antigen binding fragment thereof selected from the group consisting of full-length antibodies, Fab fragments, Fab′ fragments, F(ab′)₂ fragments, Fc fragments, Fv fragments, single chain Fv fragments, (scFv)₂ and domain antibodies.

In one embodiment, the antibody or antigen binding fragment thereof is selected from full-length antibodies, Fab fragments and scFv fragments. In one particular embodiment, said at least one antibody or antigen binding fragment thereof is a full-length antibody.

In one embodiment, the antibody or antigen binding fragment thereof is selected from the group consisting of monoclonal antibodies, human antibodies, humanized antibodies, chimeric antibodies, and antigen-binding fragments thereof.

The term “monoclonal antibodies” as used herein refers to antibodies having monovalent affinity, meaning that each antibody molecule in a sample of the monoclonal antibody binds to the same epitope on the antigen, whereas the term “polyclonal antibodies” as used herein refers to a collection of antibodies that react against a specific antigen, but in which collection there may be different antibody molecules for example identifying different epitopes on the antigen. Polyclonal antibodies are typically produced by inoculation of a suitable mammal and are purified from the mammal's serum. Monoclonal antibodies are made by identical immune cells that are clones of a unique parent cell (for example a hybridoma cell line). The term “human antibody” as used herein refers to antibodies having variable and constant regions corresponding substantially to, or derived from, antibodies obtained from human subjects. The term “chimeric antibodies” as used herein, refers to recombinant or genetically engineered antibodies, such as for example mouse monoclonal antibodies, which contain polypeptides or domains from a different species, for example human, introduced for example to reduce the antibodies' immunogenicity. The term “humanized antibodies” refers to antibodies from non-human species whose protein sequences have been modified to increase their similarity to antibody variants produced naturally in humans, for example in order to reduce immunogenicity.

Nanobody® molecules (single-domain antigen-binding fragments) allow a broad range of biotechnological and therapeutic applications due to their small size, simple production and high affinity. They have for instance been used to target specific immune cell types in mice (Groeve, K. et al. 2010 J Nucl Med 51(5): p. 782-9). Decorating the IBs described herein with affinity binders, such as Nanobody® molecules, allows targeting of the IBs to, for example, specific immune cell types, which would likely strongly increase the efficiency of the desired immune response. Nanobody® molecules can also be used to target IBs to tumors or damaged tissue. It will be appreciated that agonistic Nanobody® molecules may activate certain cells, while antagonistic Nanobody® molecules may inhibit certain processes, cells and/or components.

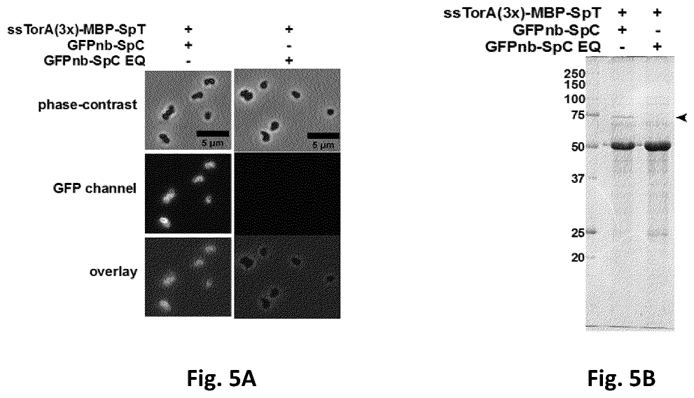

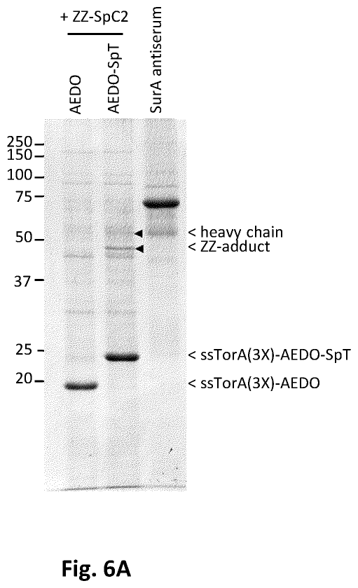

In Example 5, the inventors show that it is possible to obtain this kind of decoration by making inclusion bodies from the fusion sequence comprising three copies of the IBFS ssTorA, maltose binding protein as a model protein and a sequence expressing SpyTag, according to the construct of ssTorA(3×)-MBP-SpT. The inclusion bodies were mixed in PBS with a Nanobody® molecule having affinity for green fluorescent protein GFP (denoted GFPnb herein) already fused to the partner peptide SpyCatcher (GFPnb-SpC). As a control, ssTorA(3×)-MBP-SpT inclusion bodies in PBS were also mixed with a GFPnb fused to a catalytically inactive SpyCatcher mutant (E56Q) which lacks the ability to form an isopeptide bond with the SpyTag coupling peptide (denoted GFPnb-SpC EQ herein). The mixes were analyzed using phase contrast microscopy (FIG. 5A) and the inclusion bodies from the mix with GFPnb-SpC were uniformly fluorescent whereas those incubated with GFPnb-SpC EQ were dark. The mixes were also analyzed using SDS-PAGE followed by Coomassie staining (FIG. 5B). It was concluded that the SpyCatcher part of the GFPnb-SpC construct formed an isopeptide bond with the SpyTag of the ssTorA(3×)-MBP-SpT construct, linking the inclusion body to a GFPnb which subsequently interacts with a GFP.

### Expression Vectors

To express the fusion polypeptide of the current disclosure, the nucleic acid encoding it is constructed in the form of an expression vector or integrated into the genome of the host cell and expressed directly therefrom.

The genetic expression construct can be created using standard molecular biology techniques involving restriction enzymes, DNA ligases, PCR, oligonucleotide synthesis, DNA purification and other methods well-known to a person skilled in the art.

In one embodiment, the expression construct comprises a transcriptional unit which comprises the POI sequence and the coupling peptide (e.g. a peptide tag) sequence. Suitably, the coupling peptide (e.g. the peptide tag) sequence is located at the C-terminal end (the C-terminus) of the POI. In some embodiments, the peptide tag sequence is at the N-terminal end (the N-terminus) of the POI. In other embodiments, the peptide tag sequence is internal in the POI as hereinbefore described. Optionally, the genetic construct can further include an inclusion body forming sequence (IBFS) to facilitate inclusion body production. In this case, the transcriptional unit is arranged such that the reading frame of the portion encoding the POI matches the reading frame of the IBFS. Optionally, the transcriptional unit also comprises sequence encoding an intermediate amino acid sequence between the POI and the IBFS and/or sequence encoding additional amino acid sequences providing other functionalities, e.g. tags for purification of the POI.

In the expression construct, the transcriptional unit is arranged such that it is operably linked to one or more promoters and/or other sequences controlling its expression. Typically, the construct comprises a region 5′ of the transcriptional unit which harbors a promoter or transcription initiation region, and, optionally, a region 3′ of the transcriptional unit which controls transcription termination. Such control regions typically, although not necessarily, derive from genes that are native to the selected expression host cell.

Transcription initiation regions and promoters that are useful for driving expression of the fusion polypeptide from the transcriptional unit in different host cells are numerous and familiar to those skilled in the art. Suitable promoters depend on the host cell selected for expression. These include, but are not limited to, the tet, lac, tac, trc, ara (pBAD), trp, rha, lambda PL and T7 promoters for use in E. coli, the amy, apr and npr promoters for use in Bacillus and the CYC1, HIS3, GAL1, GAL10, ADH1, PGK, PHO5, GAPDH, ADC1, TRP1, URA3, LEU2, ENO and TPI promoters for use in Saccharomyces. Preferred promoters in E. coli include the tet, araBAD and lac and T7 promoters. Other promoters having similar kinetic properties are also preferred, both in E. coli and in other hosts. Such promoters enable strong and fast protein production and therefore contribute to efficient inclusion body production, as is described further below.

A transcription termination region may optionally be included in the vector for optimization of expression and/or increasing the stability of the transcribed mRNA. Such regions are also known in the art, or may be derived from various genes native to the preferred host.

Furthermore, the construct typically comprises other functions, such as one or more selection markers and a sequence allowing and controlling autonomous replication of the vector, e.g. an origin of replication (ori). The origin of replication determines the copy number of the vector. Preferably, the origin of replication is a high copy number origin of replication. Such origins of replication enable strong and fast protein production and therefore contribute to efficient inclusion body production, as will be described further below.

The vector is preferably an autonomously or self-replicating plasmid, a cosmid, a phage, a virus or a retrovirus. A wide variety of host/vector combinations maybe employed in expressing the fusion polypeptides of this invention. Useful expression vectors, for example, may comprise of segments of chromosomal, non-chromosomal and/or synthetic nucleic acid sequences. Suitable vectors include vectors with a specific host range, such as vectors specific for e.g. E. coli, as well as vectors with a broad host range, such as vectors useful for Gram-negative or Gram-positive bacteria.

More preferred are vectors with a specific host range, such as vectors specific for e.g. E. coli.

Other useful vectors for e.g. expression in E. coli are: pQE70, pQE60 and pQE-9 (QIAGEN, Inc.); pBluescript vectors, Phagescript vectors, pNH8A, [rho]NH16a, pNH18A, [rho]NH46A (Stratagene Cloning Systems, Inc.); ptrc99a, pKK223-3, [rho]KK233-3, pDR540, pRIT5 (Pharmacia Biotech, Inc.); pLG338, pACYC184, pBR322, pUC18, pUC19, pKC30, pRep4, pACYC177, pACYC184, pRSFIOIO and pBW22 (Wilms et al., 2001 Biotechnology and Bioengineering, 73 (2) 95-103) or derivatives thereof. Further useful plasmids are well known to the person skilled in the art and are described e.g. in “Cloning Vectors” (Eds. Pouwels P. H. et al. Elsevier, Amsterdam-New York-Oxford, 1985).

The vector is introduced in a host cell and the protein encoded by the vector is expressed in inclusion bodies. In the case of the current disclosure, this leads to inclusion bodies of the protein of interest displaying coupling peptides (e.g. on their surfaces) that are accessible to their partner peptides. The coupling peptide on the inclusion body has a specific affinity for at least one partner peptide, with which it can form an isopeptide bond. Consequently, a very robust covalent bond is formed, linking the coupling peptide directly to the partner peptide and indirectly linking the inclusion body to the partner peptide.

### Expression Kinetics for Efficient Inclusion Body Formation

Typically, although not exclusively, vectors in the present disclosure are arranged to facilitate a relatively high level of expression of the fusion polypeptide (e.g. the POI linked to the coupling peptide, optionally comprising an IBFS) in a relatively short period of time. Fast and strong expression of the fusion polypeptide contribute to efficient inclusion body formation. The expression of the fusion polypeptide in the host cell at any time is determined not only by the level, i.e. strength, of expression, but also of the rate, i.e. kinetics, of the expression. For efficient inclusion body formation, it is preferred that the level of expression is high, i.e. that the expression is strong, and also that the rate of expression is high, i.e. that the expression is fast. A high level of expression at a fast rate can be achieved in various ways. For example, a strong promoter providing rapid expression kinetics can be selected for controlling the expression of the fusion polypeptide. An alternative way is to arrange the nucleic acid sequence encoding the fusion polypeptide in a vector of a high copy number, such that multiple copies of the nucleic acid encoding the fusion polypeptide will be present in the host cell. As expression is induced, the fusion polypeptide will be expressed in parallel from the multiple copies, ensuring a high level of expression at a fast rate. A combination of a strong, fast promoter and a vector of high copy number provides even more efficient expression in terms of level and speed.

Strong and fast expression, facilitating IB formation, may be achieved by use of a vector or plasmid with a high copy number origin of replication. A high number of copies of the expression vector in the cell enables expression from a high number of transcriptional units in parallel at any one time, leading to a fast and strong expression of the fusion polypeptide.

Although fast and strong expression may be advantageous and is typically preferred, it will be appreciated that a slower rate of expression of the IBs (e.g. by using a vector with slow induction kinetics) may also be suitable.

### Host Cells

For protein expression and production of a fusion polypeptide, the vector comprising the transcriptional unit encoding the fusion polypeptide is transformed into a suitable host cell, using a suitable method known in the art. The host cell is preferably a cell which can be cultured and manipulated by methods well known to a person skilled in the art, which is able to express heterologous proteins and in which inclusion bodies may form upon overexpression of certain polypeptides. The host cell carrying the expression vector encoding the fusion polypeptide constitutes an expression system for production of the fusion polypeptide. The expression system may be inducible or non-inducible.

Preferred host cells for expression of the fusion polypeptide in inclusion bodies include cells of microbial hosts such as bacteria, yeast and filamentous fungi. Examples of host cells that may be used include, but are not limited to, species of the bacterial genera Escherichia, Salmonella, Bacillus, Pseudomonas, Erwinia, Agrobacterium, Lactococcus, Vibrio, Shigella, Burkholderia, Acinetobacter, Zymomonas, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Pantoea, Sphingomonas, Methylomonas, Methylobacter, Methylococcus, Alcaligenes, Synechocystis, Synechococcus, Anabaena, Thiobacillus, Methanobacterium, Klebsiella, Myxococcus, Bordetella and Caulobacter, the fungal or yeast genera such as Aspergillus, Trichoderma, Saccharomyces, Pichia, Yarrowia, Candida and Hansenula.

### Expression of Fusion Polypeptide

Expression of a polypeptide of interest in inclusion bodies can be achieved in a host cell. The host cell is cultured under conditions wherein the nucleic acid encoding the fusion polypeptide is translated to a multitude of fusion polypeptide molecules and the fusion polypeptide molecules aggregate in inclusion bodies.

The host cells are cultured in a culture medium that is suitable for the particular host cell. For example, the medium comprises a suitable carbon source. Furthermore, the medium is preferably optimized for protein expression. For example, the host cells may be cultured in conventional media known in the art, such as a complex medium like Luria-Bertani broth or “nutrient yeast broth medium”, a glycerol containing medium as described by Kortz et al. 1995, J Biotechnol 39, 59-65, or a mineral salt medium as described by Kulla et al. 1983, Arch Microbiol 135, 1. The medium may be modified as appropriate, e.g. by adding further ingredients such as buffers, salts, vitamins or amino acids. An antibiotic which matches the antibiotic resistance marker of the expression vector is preferably added to the medium in order to ensure stable presence of the vector and thus stable protein expression.

If the host is Escherichia coli, Luria-Bertani broth (LB medium) is advantageously used. The cells are generally cultured at a temperature of 37° C. When the culture reaches early log phase (at an OD₆₆₀ of from approximately 0.3 to approximately 0.5), expression of target protein is suitably induced by the addition of an inducer to the culture medium, the inducer being adapted to the promoter of the expression vector used. The inducer induces expression of the fusion polypeptide comprising the POI and the inclusion body forming sequence. Induction is generally performed at a temperature of about 30 to 45° C., often at a temperature of approx. 37° C. Induction at slightly higher temperatures, such as at approx. 42° C., is often preferred because it often results in more efficient inclusion body formation.

As to suitable systems for cell culture, continuous or discontinuous culture such as batch culture or fed batch culture may for example be used, in culture tubes, shake flasks or bacterial fermenters. The expression of a fusion polypeptide can be monitored by e.g. SDS-PAGE combined with Coomassie/silver staining, Western blotting or variants thereof including dot blotting.

Cell growth may also be monitored by following optical density at 600 nm or 660 nm over time. As the cell culture reaches a stage which is optimal for protein recovery, cells are harvested and the inclusion bodies containing fusion polypeptide recovered from the culture of host cells. In order to obtain a maximum yield of the expressed polypeptide, the cells are usually harvested as the cell culture reaches stationary phase. Typically, the cells are homogenized or lysed, for example by EDTA and/or lysozyme treatment, and/or sonication or French press, in order to release the insoluble inclusion bodies comprising the fusion polypeptides.

### Recovery of Inclusion Bodies

Methods for isolating inclusion bodies from cell lysates are well known in the art and include centrifugation, filtration and combinations thereof (Burgess R R 2009 Methods Enzymol 463:259-82; Nguyen L 1993 Protein Expr Purif 4:425-433; Palmer and Wingfield 2012 Curr Protoc Protein Sci Chapter 6: UNIT 6.3; Batas B et al. 1999 J Biotechnol 68(2-3):149-58). Typically, the process involves several cycles of homogenization.

Partner peptides (e.g. Catchers) and their associated additional moieties are typically, although not exclusively, cloned and expressed in a soluble form using vectors/genomes and hosts that may be different to those used to express the POI and the coupling peptides. Thus, while the partner peptides and their additional (functional) moieties may be cloned and expressed in bacterial (e.g. E. coli) or yeast cells, they may also be cloned and expressed in eukaryotic cells such as insect cells (e.g. S2, Sf9, Mimic™ Sf9, Sf21 and Trichoplusia ni High-5) or mammalian cells (e.g. PER.C6.®, COS-7, CHO, HeLa and HEK293) and purified using standard techniques and methods known to a person of skill in the art.

The decorated inclusion body may advantageously be used to deliver a specific drug to a certain target. Thus, in certain aspects and embodiments, the inclusion body, complex, nucleic acid, nucleic acid construct, and/or host cell hereinbefore described may be administered to a subject separately, or in the form of a composition. Said composition may comprise an acceptable carrier, diluent or excipient. In one embodiment, the composition is a pharmaceutical and/or immunological composition. In one embodiment, the composition is a vaccine composition.

In one embodiment, the inclusion body or complex disclosed herein is used as a medicament.

The inclusion body of the present invention may be used as a therapeutic, diagnostic, prognostic or prophylactic agent. This can, for example, be achieved by using an antibody as the additional moiety attached to the partner peptide, so that an antigen with affinity for said antibody may be detected in a human body. An example that makes use of this technology is disclosed in Example 5 and FIG. 5, wherein the additional moiety is a green fluorescent Nanobody® molecule with affinity for green fluorescent protein. By exchanging the GFP Nanobody® molecule to a Nanobody® molecule with affinity for a protein specifically present under conditions of a certain illness, the disclosed inclusion body can be used to diagnose and/or provide a prognosis for diseases.

Some aspects and embodiments of the invention also provide methods of treatment, diagnosis, prognosis or prevention of a disease or disorder in a subject comprising the step of administering the inclusion body, complex or composition of the invention to said subject. The subject may be any animal, such as a mammal selected from a human, a farm animal (e.g. cattle, sheep, pig and goat) and a companion animal (e.g. horse, dog and cat). The animal may also be a fish (e.g. salmon, trout, seabass, tilapia and catfish) or a bird (e.g. poultry).

The inclusion body, complex or composition according to the embodiments previously disclosed may also be used as a vaccine. By linking for example immunostimulative moieties to the partner peptide and thus to a POI expressed in the form of an antibody, the disclosed inclusion body may be used to stimulate the immune system such that it targets a specific type of diseased cells or an infection.

Targeting moieties of the kind presented above provide a way to create decorated inclusion bodies with affinity for specific components of the immune system. Further, by combining the ability to aggregate proteins of interest such as for example antigens in inclusion bodies with decorating said inclusion bodies with immune targeting moieties, the inventors have made it possible to stimulate a certain response from the immune system and using a decorated inclusion body as it is described herein as, for example, a vaccine. Based on the same principle, the current disclosure provides a drug delivery system wherein the inclusion body consists of aggregated biological drug molecules and is decorated with a targeting moiety with affinity for a specific cell type, such as for example a cancer cell, thus using the inclusion body as a medicament. Targeted drug delivery is greatly desired as it may provide an administrational route which efficiently delivers the drug to the site and/or target of interest and is less straining on the patient compared to for example oral administration where the drug is distributed through the blood system.

In view of the foregoing, a skilled person will understand that this approach may be of use for applications in which specific interactions between IBs and cells or tissues in humans and animals are a prerequisite. For example, immunization methods involving the use of IBs for antigen delivery are expected to benefit significantly from the decoration of IBs with affinity molecules (e.g. antibodies, Affibody® molecules or Nanobody® molecules) targeting specific subsets of cells of the immune system, allowing more tailored immune responses. Similarly, decoration of the IBs with affinity molecules recognizing tumor-specific molecules (e.g. molecules present on the surface of tumor cells) would allow for targeted IB-based cancer therapy.

Moreover, proteinaceous adhesins or glycans may be attached to the surface of IBs to facilitate their association with preferred cell types or tissues. Functionality of IBs for antigen delivery or cancer treatment may also be enhanced by equipping them with immuno-modulatory molecules, such as adjuvants or cytokines, to allow for customized immune responses.

In other embodiments, the IBs may be decorated with binding partners that allow immobilization of IBs on specific matrices, e.g. to facilitate immobilized biocatalysis.

It will be appreciated that the inclusion bodies, complexes and compositions disclosed herein may be suitable in various medical and/or veterinary applications and may be used to treat any animal, such as a mammal, e.g. a human, a farm animal (e.g. cattle, sheep, pig or goat) or a companion animal (e.g. horse, dog or cat). In one embodiment, said mammal is a human. The animal may also be a bird (e.g. poultry) or a fish (e.g. salmon, trout, seabass, tilapia or catfish).

In conclusion, described herein is a universal, easy and cost-efficient method for decorating inclusion bodies with functional moieties using a ligation system which makes said inclusion bodies applicable for a variety of biotechnical and biomedicinal purposes.

So that the invention may be fully understood and put into practical effect, reference is made to the following non-limiting Examples.

### EXAMPLES

### General Procedures

### Protein Expression and Purification of Soluble Partner Peptide Comprising Additional Moieties

E. coli BL21 (DE3) cells were used to harbor the different constructs disclosed herein, cloned into the appropriate expression vector (pET28a or pDEST14). The cells were left overnight and the overnight culture were subcultured in fresh medium and cell growth was continued. Upon reaching early log phase (OD₆₀₀≈0.3), expression of the protein of interest was induced with 0.4 mM of IPTG. Cells were collected from the culture 4 h after induction by low speed centrifugation and resuspended in 45 ml PBS. Thereafter, cells were again collected by low speed centrifugation and stored at −80° C. The cells were resuspended (42× concentrated) in binding buffer (50 mM sodium phosphate, 300 mM NaCl, pH 7.4). PMSF was added to 0.5 mM. The cell suspension was passed twice through a OneShot cell disruptor at 1.2 kbar. Subsequently, the suspension was cleared through two centrifugation steps at 4° C.: the first step at 10,000×g for 10 min and the second at 293,000×g for 45 min. The protein of interest was purified from the cleared lysate using TALON Superflow (GE Healthcare Life Sciences) according to the manufacturer's instructions.

### Inclusion Body Expression and Isolation of POI Comprising Coupling Peptide

E. coli TOP10F′ cells harboring constructs cloned into the expression vector pIBA from overnight culture were subcultured in fresh medium and continued to grow until reaching early log phase (OD₆₀₀≈0.3), whereby the expression of fusion protein was induced with 0.2 μg/ml of anhydrotetracycline. Cells were collected from the culture 3 h after induction through low speed centrifugation and then resuspended in 40 ml of 10 mM Tris-HCl of pH 8.0. Cells were again collected by low speed centrifugation and stored frozen (−20° C. or −80° C.). The cells were subsequently resuspended in 10 mM Tris-HCl pH 8.0, 1 mM Na-EDTA, 10 μg/ml lysozyme to an OD₆₀₀ of 20 (as calculated from final culture OD) and incubated for 1 h at 37° C. The suspension was cooled on ice and sonicated using a tip sonicator (Branson Sonifier 250, settings varied per sample) until all cells were lysed (cell lysis verified by microscopy). All the insoluble material including inclusion bodies were collected using centrifugation at 15,000×g for 15 min. The pelleted material was resuspended in a half volume of 10 mM Tris-HCl pH 8.0 and sonicated to break up clumps of inclusion bodies. A half volume of 10 mM Tris-HCl pH 8.0, 2 mM Na-EDTA, 2% Triton X-100 (to give 1 mM EDTA and 1% TX-100 final) was added. The suspension was incubated agitated for 1 h at ambient temperature. Inclusion bodies were collected through centrifugation at 15,000×g for 15 min, again resuspended in a half volume of 10 mM Tris-HCl pH 8.0 and sonicated to break up clumps. A half volume of 10 mM Tris-HCl pH 8.0, 2 M urea was added. The suspension was incubated agitated for 1 h at ambient temperature. Inclusion bodies were collected using centrifugation at 15,000×g for 15 min, resuspended in a half volume of 10 mM Tris-HCl pH 8.0 and sonicated to break up clumps. A half volume of 10 mM Tris-HCl pH 8.0, 2 M NaCl was added. The suspension was incubated agitated for 1 h at ambient temperature. Inclusion bodies were collected using centrifugation at 15,000×g for 15 min, resuspended in one volume of 10 mM Tris-HCl pH 8.0 and sonicated to break up clumps. Inclusion bodies were collected using centrifugation at 15,000×g for 15 min, resuspended in PBS and stored at −20° C.

### Example 1

### Proof of Concept

This example illustrates proof of concept for the decoration of IBs with functional affinity moieties to permit targeting of the IBs to specific cells and/or tissues. FIG. 1A is a schematic drawing of an inclusion body (IB) decorated with a Nanobody® (GFPnb) with affinity for green fluorescent protein (GFP) using the ligation system of SpyTag and SpyCatcher. The GFPnb is able to bind GFP, resulting in fluorescently decorated IBs. FIG. 1B is a ribbon diagram showing the partner peptide SpyCatcher (left side) and the GFPnb bound to GFP (right side).

### Example 2

### Conjugation to Inclusion Bodies Using Two Different Isopeptide Bonding Technologies

This example illustrates the successful covalent conjugation of two different coupling peptides (i.e. peptide tags) to their respective cognate partner peptides (i.e. binding protein partners) when fused to a protein expressed in IB form. The sequence ssTorA(3×)-MBP was genetically fused with either a C-terminal SpyTag (ssTorA(3×)-MBP-SpT; SEQ ID NO:2) or SnoopTag (ssTorA(3×)-MBP-SnT; SEQ ID NO:13) and the resulting fusions were expressed in IB form in two different batches.

A soluble fusion protein comprising an N-terminal SpyCatcher component and a C-terminal SnoopCatcher component (SpyCatcher-SnoopCatcher; SpC-SnC; SEQ ID NO:1) was expressed from pET28a. Purified SpyCatcher-SnoopCatcher was dialyzed against 500× the volume of PBS using dialysis membrane with a 3500 Da MWCO (Spectra/Por) for 16h at 4° C. After dialysis glycerol was added to 10% (v/v) final concentration.

Inclusion bodies isolated from cells expressing ssTorA(3×)-MBP-SpT or ssTorA(3×)-MBP-SnT (˜15 μg total protein) were mixed with the purified and dialyzed SpyCatcher-SnoopCatcher (10 μM final concentration) in PBS pH 8.0. The mixtures were incubated at 25° C. for 2h.

Successful conjugation of SpyCatcher-SnoopCatcher to ssTorA(3×)-MBP-SpT IBs and ssTorA(3×)-MBP-SnT IBs can be seen from SDS-PAGE and Coomassie staining analysis of samples corresponding to 0.75 μg of inclusion body. Adducts of −75 kDa are efficiently formed, indicating covalent linkage between the peptide tag (SpyTag or SnoopTag) and the binding protein partner (SpyCatcher or SnoopCatcher), seen in FIG. 2A and FIG. 2B respectively.

For comparison, equal amounts of inclusion body material and SpyCatcher-SnoopCatcher as used in the reaction mix were loaded on the gel. Molecular mass (kDa) markers are indicated at the left side of the panels. The adducts are indicated with arrowheads.

### Example 3

### Conjugation to ssTorA(3×)-Induced and ssTorA(3×)-Independent Inclusion Bodies Using Two Different Isopeptide Bonding Technologies

This example shows conjugation of partner proteins to IBs using the two different isopeptide bonding systems SpyCatcher/SpyTag and SnoopCatcher/SnoopTag. Furthermore, it illustrates that both systems may be used for conjugation to IB forming sequences of various designs. Moreover, it shows successful coupling of partner proteins to Pla2 IBs that were formed independent of an IB-formation tag.

Fusion protein SpyCatcher-SnoopCatcher (SpC-SnC; SEQ ID NO:1) was expressed from vector pET28a as in Example 2 and dialyzed against 500× the volume of PBS using dialysis membrane with a 3500 Da MWCO (Spectra/Por) for 16h at 4° C. After dialysis glycerol was added to 10% (v/v) final concentration.

Inclusion bodies derived from Pla2 carrying either a C-terminal SpyTag or SnoopTag were produced as constructs Pla2-SpT (SEQ ID NO:4) and Pla2-SnT (SEQ ID NO:3), respectively. Likewise, inclusion bodies were made comprising short antigenic epitopes of different origin (AEDO) fused between IBFS ssTorA(3×) and SnoopTag or SpyTag as constructs ssTorA(3×)-AEDO-SnT and ssTorA(3×)-AEDO-SpT. The inclusion bodies (30 μg total protein) were mixed with purified SpyCatcher-SnoopCatcher (70 μM final concentration) in PBS. The mixtures were incubated at 25° C. for 2h. The inclusion bodies were collected through centrifugation and resuspended in PBS. Samples corresponding to 1.5 μg of inclusion body total protein were analyzed using SDS-PAGE and Coomassie staining. Analysis shows that IBs derived from Pla2 carrying either a C-terminal SpyTag or SnoopTag were covalently coupled to SpyCatcher-SnoopCatcher fusion protein when mixed together, as is verified by the adducts visible in FIG. 3A (indicated by arrowheads). Likewise, ssTorA(3×)-AEDO-SnT or ssTorA(3×)-AEDO-SpT IBs were covalently coupled to SpyCatcher-SnoopCatcher, as is verified by the adducts visible in FIG. 3A.

Further verification was done by SDS-PAGE and Western blotting using samples corresponding to 1.5 μg of inclusion body total protein and monoclonal anti-polyhistidine antibody (Sigma, H1029) (FIG. 3B). Molecular mass (kDa) markers are indicated at the left side of the panels. In FIG. 3A the adducts are indicated with arrowheads; in FIG. 3B the corresponding positions are likewise indicated.

### Example 4

### Conjugation of SnT-mEGFP-SpT to ssTorA(3×)-MBP-KT Inclusion Bodies

This example illustrates the successful conjugation of biologically functional molecules to inclusion bodies via covalent isopeptide bond formation between a partner peptide (SpyTag) and a coupling peptide. In this case the coupling peptide was KTag genetically fused to maltose binding protein ssTorA(3×)-MBP at its C-terminus (ssTorA(3×)-MBP-KT; SEQ ID NO:5). The inclusion bodies were isolated from cells expressing ssTorA(3×)-MBP-KT (14 μg total protein).

A monomeric enhanced green fluorescent protein (mEGFP) was used as the additional moiety coupled to the partner peptide (SpyTag) on the C-terminus and a redundant SnoopTag at the N-terminus (SnT-mEGFP-SpT; SEQ ID NO:7). SnT-mEGFP-SpT was expressed from pET28a and purified. Ligase protein SpyLigase (SEQ ID NO:6) was present to drive the coupling reaction and was expressed from pDEST14. SpyLigase and SnT-mEGFP-SpT were dialyzed against 250× the volume of PBS using dialysis membrane with a 3500 Da MWCO (Spectra/Por) for 3h, 15h and 3h at 4° C. with PBS exchange inbetween.

The inclusion bodies isolated from cells expressing ssTorA(3×)-MBP-KT (14 μg total protein) were mixed with purified SnT-mEGFP-SpT and SpyLigase (5 μM and 20 μM final concentration respectively) in 40 mM Na₂HPO₄, 20 mM citric acid, 1.5 M trimethylamine N-oxide (TMAO). In the control experiment, PBS was added instead of SpyLigase. The mixtures were incubated at 4° C. for 23 h. The inclusion bodies were collected by centrifugation and resuspended in PBS.

Successful conjugation of SnT-mEGFP-SpT to ssTorA(3×)-MBP-KT IBs according to ssTorA(3×)-MBP-KTag-SpT-mEGFP-SnT was verified through fluorescence microscopy analysis which is shown in FIG. 4A (scale bar indicates 1 pm). The analysis shows GFP fluorescence signals emitted by IBs mixed with SnT-mEGFP-SpT and SpyLigase, whereas no signals are detected when SpyLigase is absent.

Samples corresponding to 0.7 μg of inclusion body total protein were analyzed by SDS-PAGE and Coomassie staining as seen in FIG. 4B where molecular mass (kDa) markers are indicated at the left side of the panel. The ssTorA(3×)-MBP-KT-SpT-mEGFP-SnT adduct is indicated with an arrowhead. The analysis discloses the emergence of a band representing an adduct between ssTorA(3×)-MBP-KT and SnT-mEGFP-SpT when SpyLigase is present, demonstrating covalent isopeptide bond formation between ssTorA(3×)-MBP-KT IBs and soluble SnT-mEGFP-SpT.

### Example 5

### Conjugation of GFPNanobody®-SpyCatcher (GFPnb-SpC) to ssTorA(3×)-MBP-SpT Inclusion Bodies

This example, which is an extension of the proof of concept in Example 1, illustrates successful use of ligation system technology for coupling of additional moieties to inclusion bodies. More specifically, the example shows the coupling of Nanobody® molecules to inclusion bodies.

E. coli BL21 (DE3) cells was used to harbor the construct of GFPnb-SpyCatcher (SEQ ID NO:8) cloned into vector pET22b in an overnight culture. The culture was then subcultured in fresh medium and cell growth was resumed. When the culture reached OD₆₀₀≈0.85, expression of the GFPnb was induced with 0.5 mM of IPTG and the temperature was lowered to 12° C. The cells were collected from the culture 20 h after induction by low speed centrifugation and stored at −20° C. The cells were then thawed and resuspended in PBS. After 30 min of incubation at 21° C. with shaking the cells were removed using centrifugation. As a control, also a construct containing SpyCatcher with an amino acid substitution E77Q, which disrupts isopeptide bond formation, was produced (GFPnb-SpC EQ; SEQ ID NO:9).

The GFPnb-SpC and GFPnb-SpC EQ proteins were purified from the suspending medium using TALON Superflow (GE Healthcare Life Sciences) according to the manufacturer's instructions. Purified GFPnb-SpC and GFPnb-SpC EQ were dialyzed against 1000× the volume of PBS using dialysis membrane with a 3500 Da MWCO (Spectra/Por) for 16h at 4° C.

Inclusion bodies isolated from cells expressing ssTorA(3×)-MBP-SpT (SEQ ID NO:2) (˜15 μg total protein) were mixed with purified GFPnb-SpC or GFPnb-SpC EQ (3.0 μM or 3.2 μM final concentration respectively) in PBS. The mixtures were incubated at 25° C. for 2h. The inclusion bodies were collected through centrifugation and resuspended in PBS. Purified GFP (SEQ ID NO:10) expressed from pET28a was added to 2.8 μM final concentration.

Successful conjugation of GFPnb-SpC to ssTorA(3×)-MBP-SpT IBs follows from SDS-PAGE and Coomassie staining analysis. Adducts of −75 kDa adduct are efficiently formed, indicating covalent linkage between the coupling peptide/peptide tag (SpC) and the partner peptide/binding protein partner (SpT), seen in FIG. 5B. As a control, no adduct was observed upon incubation of ssTorA(3×)-MBP-SpT IBs with GFPnb-SpC EQ carrying a SpyCatcher moiety deficient in isopeptide bonding formation.

To confirm functionality of the Nanobody® molecule upon the conjugation reaction, IBs were incubated with soluble GFP for 50 min at ambient temperature and the inclusion bodies were collected using centrifugation and thereafter resuspended in PBS and analyzed by fluorescence microscopy.

Effective conjugation of functional Nanobody® molecules to the IBs was demonstrated by the emission of a fluorescent signal by IBs incubated with GFPnb-SpC and subsequently treated with GFP (FIG. 5A). As a control, no fluorescence signal was detected upon GFP treatment of IBs incubated with GFPnb-SpC EQ. Hence, isopeptide bond formation between ssTorA(3×)-MBP-SpT IBs and GFPnb-SpC (ssTorA(3×)-MBP-SpT-SpC-GFPnb) must occur to achieve fluorescent labeling of the IBs in the assay used.

### Example 6

### Coupling of a Functional Antibody-Binding ZZ Molecule to IBs

This example illustrates that isopeptide bonding technology can be used to decorate IBs with antibodies through coupling of Ig-binding proteins or derived domains. Also, functionality of SpyCatcher002 in the context of IBs is demonstrated.

Staphylococcal Protein A, streptococcal Protein G and Peptostreptococcus protein L are proteins that bind to mammalian immunoglobulin molecules. Recombinant versions are also available, and are widely used as affinity molecules in antibody purification procedures. Examples of such molecules also include the derivative of antibody-binding domain B of Protein A called protein Z (Nilsson et al (1987), Prot Eng 1:107-113), or a tandem-fused dual version called ZZ. Like Protein A, Z and ZZ have affinity for the Fc domain of antibodies. Another example is Protein NG, a recombinant fusion protein that combines IgG binding domains of both Staphylococcus aureus Protein A and streptococcal Protein G, and has a mass of 50 kDa. Whereas Z and ZZ mainly bind human and rabbit IgGs and some classes of mouse and rat IgGs, Protein NG binds to all subclasses of human, rabbit, mouse and rat IgGs.

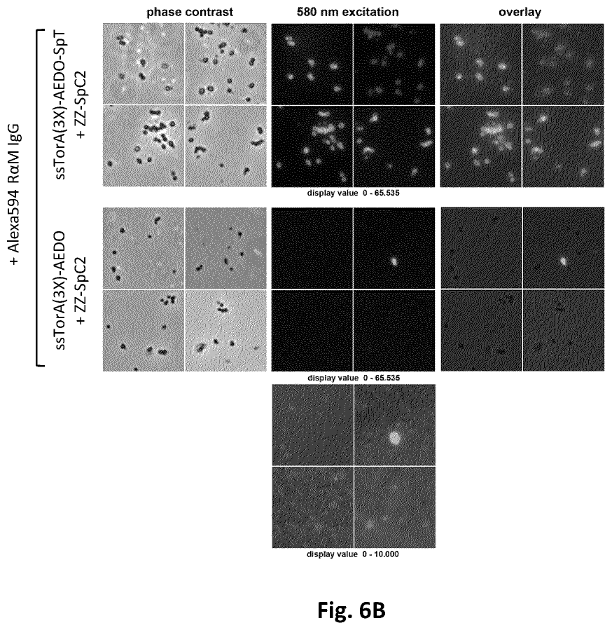

To allow isopeptide bond-mediated coupling of ZZ to IBs, a fusion protein was created and purified comprising ZZ and a C-terminally located SpyCatcher002 moiety (ZZ-SpC2; SEQ ID NO:11). Furthermore, ssTorA(3×)-AEDO-SpT IBs were prepared carrying a cognate SpT moiety. ZZ-SpC2 and the IBs were mixed and incubated at 4° C. overnight. As a control ZZ-SpC2 was also mixed with ssTorA(3×)-AEDO lacking a SpT. Next morning, IBs were collected by low-speed centrifugation and resuspended in PBS/glycerol (15%). At this point, rabbit antiserum with polyclonal antibodies against E. coli SurA were added and the mixtures were incubated at ambient temperature for 1 hour to allow binding of the antibodies by ZZ. IBs were then isolated by low-speed centrifugation to separate IB-bound antibodies from soluble antibodies and analyzed by Coomassie stained SDS-PAGE. Successful coupling of ZZ-SpC2 to the SpT-carrying IBs was demonstrated by the appearance of a protein band representing an adduct ssTorA(3×)-AEDO-SpT and ZZ-SpC2. No adduct was observed when IBs lacking SpT were used. In turn, successful binding of SurA antibodies to IBs displaying ZZ followed from the recovery of IgG heavy chain material specifically in IB samples equipped with an SpT and decorated with ZZ-SpC2 (FIG. 6A).

Successful binding of antibodies was also demonstrated via an alternative approach, involving fluorescence microscopy (FIG. 6B). To this end, ssTorA(3×)-AEDO-SpT and ssTorA(3×)-AEDO IBs pre-incubated with ZZ-SpC2 as above, were incubated with fluorescent Alexa 594 Rabbit anti-Mouse IgGs at ambient temperature for 30 min. Subsequently, IBs were subjected to fluorescence microscopy analysis. Whereas, only background levels of fluoresce were observed for IBs lacking SpT, efficient labeling with fluorescent antibodies was observed for ssTorA(3×)-AEDO equipped with SpT and able to form covalent bonds with ZZ-SpC2 (see above).

### Example 7

### Specific Antibody Binding to IBs Through Coupling of ZZ or Protein A/G

This example illustrates that isopeptide bonding technology can be used to decorate IBs with antibodies through coupling of Ig-binding proteins or derived domains like ZZ or Protein A/G. Furthermore, retained functionality and specificity of coupled ZZ and Protein A/G at the IB surface is demonstrated.

Whereas ZZ generally binds human IgGs but only has moderate affinity for rat IgGs, Protein NG binds to both human and rat IgGs with high affinity. To allow coupling of ZZ and Protein NG to IBs, they were translationally fused to C-terminal SpyCatcher002 (SpC2) domain and purified (ZZ-SpC2 (SEQ ID NO.11) and AG-SpC2 (SEQ ID NO:12), respectively). Moreover, ssTorA(3×)-AEDO-SpT IBs were prepared with or without (mock) a cognate SpT moiety. The respective IBs were mixed with either ZZ-SpC2 and AG-SpC2 and incubated at 4° C. overnight. The next morning, IBs were isolated by centrifugation and resuspended in PBS/glycerol (15%). Suspensions were split and one half was incubated with IgGs from rat serum (Sigma 18015) and the other with IgGs from human serum for 30 min at room temperature. Subsequently, the IBs were analyzed for successful coupling of ZZ and Protein A/G and subsequent antibody binding by SDS-PAGE and Coomassie staining.

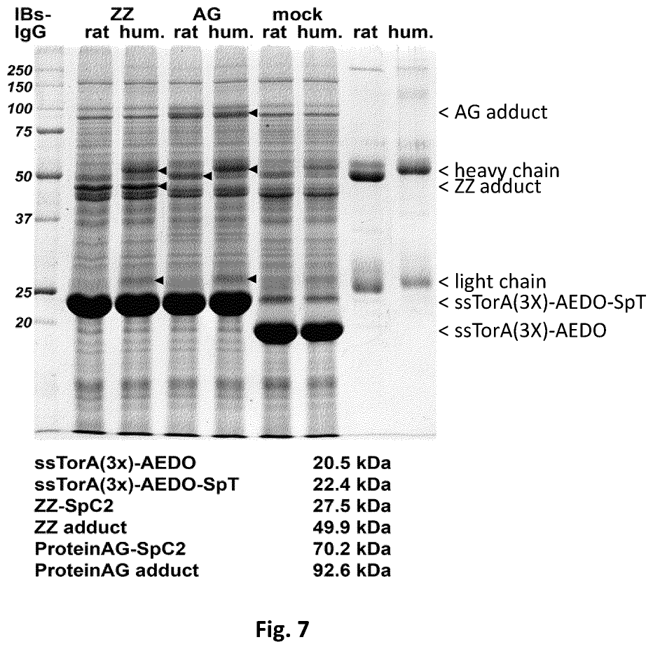

Successful covalent coupling of SpC2-equipped ZZ and Protein A/G to SpT-carrying IBs was demonstrated by the detection of adducts (ZZ adduct, AG adduct) comprising covalent fusions between ssTorA(3×)-AEDO-SpT and either ZZ-SpC2 or AG-SpC2 respectively. Functionality of ZZ when coupled to IBs was demonstrated by the appearance of Coomassie stained bands corresponding to the heavy and light chains of human antibodies, whereas no heavy or light chain material was observed in IB samples incubated with rat antibodies. IBs coupled to Protein NG displayed heavy chain and light chain bands upon incubation with IgGs of both human and rat origin, demonstrating functional association of coupled Protein NG with these two types of antibodies in the context of IBs (FIG. 7).

### Example 8

### Successful Coupling of a Binding Partner Protein to IBs in a Bacterial Lysate

This example illustrates successful conjugation of IBs to a binding partner protein presented in a bacterial lysate. Surprisingly, conjugation proceeds with a similar efficiency compared to the use of a purified conjugation partner.

Purification of binding partner proteins before linkage to IBs is a time-demanding and costly process. Carrying out the isopeptide bonding reaction with unpurified material presented in the lysate of a bacterial host strain expressing the binding partner protein construct enhances the production process of conjugated IBs in terms of time- and cost-efficiency. As proof of concept, IBs derived of fusion protein ssTorA(3×)-AEDO-SpT, carrying a C-terminal SpyTag, were conjugated to ZZ-SpC2, equipped with a C-terminal SpyCatcher002 moiety, presented in the lysate of a producing E. coli strain.

Lysates were prepared from a culture of E. coli BL21(DE3)::pET28-ZZ-SpC2 that was induced for 3h with 0.5 mM IPTG. Approximately 1,700 OD600 units of cell material was harvested and resuspended in 9 ml of binding buffer (50 mM sodium phosphate, 300 mM sodium chloride, pH7.4). Cells were lysed by passage through a OneShot cell disrupter (Constant systems Ltd., Daventry, UK) and the lysate was subjected to consecutive low-speed (10,000×g, 10 min, 4° C.) and high-speed (293,000×g, 1 h, 4° C.) centrifugation steps to remove debris and other insoluble components. The resulting cleared lysate (supernatant) was used for coupling experiments. To test conjugation, 6.1 μl of PBS-suspended ssTorA(3×)-AEDO-SpT IBs (8 μg) were mixed with 1 μl of cleared lysate containing ZZ-SpC2 and incubated at room temperature for 2h. Samples were subjected to low-speed centrifugation and the resulting supernatant (sup), containing non IB-associated material, and pellet, containing IBs and associated material, were analyzed by SDS-PAGE and Coomassie staining. As a negative control, IBs lacking a SpyTag (ssTorA(3×)-AEDO) were tested for conjugation to lysate-borne ZZ-SpC2. Moreover, reaction samples of ssTorA(3×)-AEDO-SpT and ssTorA(3×)-AEDO IBs incubated with purified ZZ-SpC2, as outlined in example 6, were analyzed for comparison.

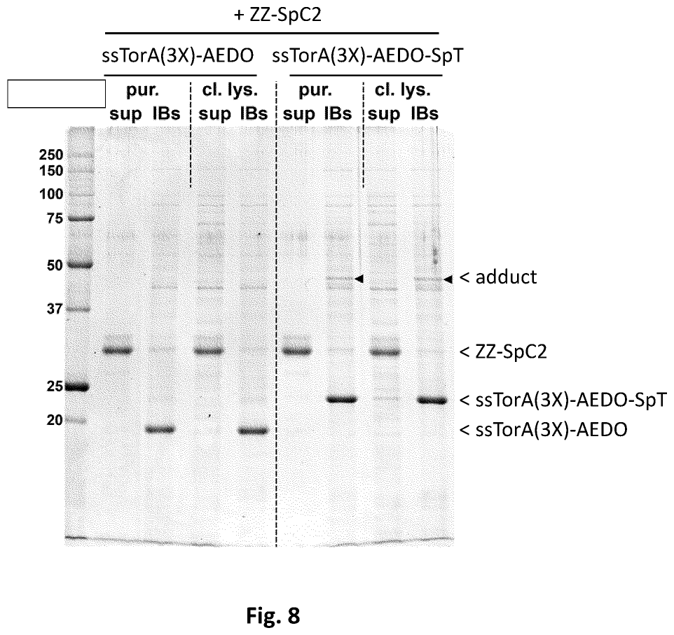

Successful conjugation of ZZ-SpC2 from bacterial lysate to ssTorA(3×)-AEDO-SpT follows the detection of an adduct (adduct) in the IB fraction comprising a covalent fusions between ssTorA(3×)-AEDO-SpT and ZZ-SpC2. No such adduct was detected in the negative control sample in which no SpyTag was available. Importantly, the intensity of the adduct band was on par with the adduct detected upon incubation of ssTorA(3×)-AEDO-SpT IBs with affinity purified ZZ-SpC2, unexpectedly indicating that use of a partner peptide (binding partner protein) in a complex lysate does not interfere with its conjugation to IBs. Moreover, highly similar protein profiles were observed in IB samples incubated with either crude or purified ZZ-SpC2 indicating that conjugation in a complex lysate environment does not lead to a higher degree of protein contamination of IB-conjugates compared to the use of purified binding partner protein (FIG. 8). Together, the data demonstrate potential for the use of non-purified binding partner proteins in the production of isopeptide bond-based IB conjugates.

Protein sequences of the constructs used to practise the invention are listed in Table 3.

TABLE 3
Protein sequences of constructs
(SEQ ID Nos. 1-13)

SpyCatcher-SnoopCatcher (SpC-SnC)
(SEQ ID NO: 1)
MSYYHHHHHHDYDSATHIKFSKRDEDGKELAGATMELRDSSGKTISTW
ISDGQVKDFYLYPGKYTFVETAAPDGYEVATAITFTVNEQGQVTVNGK
ATKGDAHIGSPANLKALEAQKQKEQRQAAEELANAKKLKEQLEKGSHM
KPLRGAVFSLQKQHPDYPDIYGAIDQNGTYQNVRTGEDGKLTFKNLSD
GKYRLFENSEPAGYKPVQNKPIVAFQIVNGEVRDVTSIVPQDIPATYE
FTNGKHYITNEPIPPK*
ssTorA(3X)-MBP-SpT
(SEQ ID NO: 2)
MNNNDLFQASRRRFLAQLGGLTVAGMLGPSLLTPRRASMNNNDLFQAS
RRRFLAQLGGLTVAGMLGPSLLTPRRASMNNNDLFQASRRRFLAQLGG
LTVAGMLGPSLLTPRRASAKIEEGKLVIWINGDKGYNGLAEVGKKFEK
DTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNP
PKTWEEIPALDKELKAKGKSALMFNLQEPYFTWPLIAADGGYAFKYEN
GKYDIKDVGVDNAGAKAGLTFLVDLIKNKHMNADTDYSIAEAAFNKGE
TAMTINGPWAWSNIDTSKVNYGVTVLPTFKGQPSKPFVGVLSAGINAA
SPNKELAKEFLENYLLTDEGLEAVNKDKPLGAVALKSYEEELAKDPRI
AATMENAQKGEIMPNIPQMSAFWYAVRTAVINAASGRQTVDEALKDAQ
TRITKGSGSGSAHIVMVDAYKPTK*
Pla2-SnT
(SEQ ID NO: 3)
MGLLDLKSMIEKVTGKNALTNYGFYGCYCGWGGRGTPKDGTDWCCWAH
DHCYGRLEEKGCNIRTQSYKYRFAWGVVTCEPGPFCHVNLCACDRKLV
YCLKRNLRSYNPQYQYFPNILCSGTGSGSKLGDIEFIKVNK*
Pla2-SpT
(SEQ ID NO: 4)
MGLLDLKSMIEKVTGKNALTNYGFYGCYCGWGGRGTPKDGTDWCCWAH
DHCYGRLEEKGCNIRTQSYKYRFAWGVVTCEPGPFCHVNLCACDRKLV
YCLKRNLRSYNPQYQYFPNILCSGTGSGSAHIVMVDAYKPTK*
ssTorA(3X)-MBP-KT
(SEQ ID NO: 5)
MNNNDLFQASRRRFLAQLGGLTVAGMLGPSLLTPRRASMNNNDLFQAS
RRRFLAQLGGLTVAGMLGPSLLTPRRASMNNNDLFQASRRRFLAQLGG
LTVAGMLGPSLLTPRRASAKIEEGKLVIWINGDKGYNGLAEVGKKFEK
DTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNP
PKTWEEIPALDKELKAKGKSALMFNLQEPYFTWPLIAADGGYAFKYEN
GKYDIKDVGVDNAGAKAGLTFLVDLIKNKHMNADTDYSIAEAAFNKGE
TAMTINGPWAWSNIDTSKVNYGVTVLPTFKGQPSKPFVGVLSAGINAA
SPNKELAKEFLENYLLTDEGLEAVNKDKPLGAVALKSYEEELAKDPRI
AATMENAQKGEIMPNIPQMSAFWYAVRTAVINAASGRQTVDEALKDAQ
TRITKGSGSGSATHIKFSKRDGY*
SpyLigase
(SEQ ID NO: 6)
MSYYHHHHHHDYDGQSGDGKELAGATMELRDSSGKTISTWISDGQVKD
FYLYPGKYTFVETAAPDGYEVATAITFTVNEQGQVTVNGKATKGGSGG
SGGSGEDSATHI*
SnT-mEGFP-SpT
(SEQ ID NO: 7)
MGSSHHHHHHSSGLVPRGSHMGKLGDIEFIKVNKGSGESGSGVSKGEE
LFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKD
DGNYKTRAEVKFEGDTLVNRIELKGIDFKEDGNILGHKLEYNYNSHNV
YIMADKQKNGIKVNFKIRHNIEDGSVQLADHYQQNTPIGDGPVLLPDN
HYLSTQSKLSKDPNEKRDHMVLLEFVTAAGITLGMDELYKGSGEGSGS
GSGSAHIVMVDAYKPTK*
GFPnb-SpC
(SEQ ID NO: 8)
MKYLLPTAAAGLLLLAAQPAMAMVQLVESGGALVQPGGSLRLSCAASG
FPVNRYSMRWYRQAPGKEREWVAGMSSAGDRSSYEDSVKGRFTISRDD
ARNTVYLQMNSLKPEDTAVYYCNVNVGFEYWGQGTQVTVSSKGSGGTG
DSATHIKFSKRDEDGKELAGATMELRDSSGKTISTWISDGQVKDFYLY
PGKYTFVETAAPDGYEVATAITFTVNEQGQVTVNGKATKGDAHIGSGH
HHHHH*
GFPnb-SpC EQ
(SEQ ID NO: 9)
MKYLLPTAAAGLLLLAAQPAMAMVQLVESGGALVQPGGSLRLSCAASG
FPVNRYSMRWYRQAPGKEREWVAGMSSAGDRSSYEDSVKGRFTISRDD
ARNTVYLQMNSLKPEDTAVYYCNVNVGFEYWGQGTQVTVSSKGSGGTG
DSATHIKFSKRDEDGKELAGATMELRDSSGKTISTWISDGQVKDFYLY
PGKYTFVQTAAPDGYEVATAITFTVNEQGQVTVNGKATKGDAHIGSGH
HHHHH*
GFP
(SEQ ID NO: 10)
MSKGEELFTGVVPILVELDGDVNGHKFSVSGEGEGDATYGKLTLKFIC
TTGKLPVPWPTLVTTLTYGVQCFSRYPDHMKRHDFFKSAMPEGYVQER
TISFKDDGNYKTRAEVKFEGDTLVNRIELKGIDFKEDGNILGHKLEYN
YNSHNVYITADKQKNGIKANFKIRHNIEDGSVQLADHYQQNTPIGDGP
VLLPDNHYLSTQSALSKDPNEKRDHMVLLEFVTAAGITHGMDELYKKL
AAALEHHHHHH*
ZZ-SpC2
(SEQ ID NO: 11)
MGKGSGSVDNKFNKEQQNAFYEILHLPNLNEEQRNAFIQSLKDDPSQS
ANLLAEAKKLNDAQAPKVDNKFNKEQQNAFYEILHLPNLNEEQRNAFI
QSLKDDPSQSANLLAEAKKLNDAQAPKGSVTTLSGLSGEQGPSGDMTT
EEDSATHIKFSKRDEDGRELAGATMELRDSSGKTISTWISDGHVKDFY
LYPGKYTFVETAAPDGYEVATAITFTVNEQGQVTVNGEATKGDAHTGS
SGSLEHHHHHH*
AG-SpC2
(SEQ ID NO: 12)
MGNAAQHDEAQQNAFYQVLNMPNLNADQRNGFIQSLKDDPSQSANVLG
EAQKLNDSQAPKADAQQNNFNKDQQSAFYEILNMPNLNEAQRNGFIQS
LKDDPSQSTNVLGEAKKLNESQAPKADNNFNKEQQNAFYEILNMPNLN
EEQRNGFIQSLKDDPSQSANLLSEAKKLNESQAPKADNKFNKEQQNAF
YEILHLPNLNEEQRNGFIQSLKDDPSQSANLLAEAKKLNDAQAPKADN
KFNKEQQNAFYEILHLPNLTEEQRNGFIQSLKDDPSVSKEILAEAKKL
NDAQAPKEEDSLEGSGSGTYKLILNGKTLKGETTTEAVDAATAEKVFK
QYANDNGVDGEVVTYDDATKTFTVTEKPEVIDASELTPAVTTYKLVIN
GKTLKGETTTEAVDAATAEKVFKQYANDNGVDGEWTYDDATKTFTVTE
KPEVIDASELTPAVTTYKLVINGKTLKGETTTKAVDAETAEKAFKQYA
NDNGVDGVWTYDDATKTFTVTEKLAAAGTGSGEGSGSVTTLSGLSGEQ
GPSGDMTTEEDSATHIKFSKRDEDGRELAGATMELRDSSGKTISTWIS
DGHVKDFYLYPGKYTFVETAAPDGYEVATAITFTVNEQGQVTVNGEAT
KGDAHTGSSGSLEHHHHHH*
ssTorA(3X)-MBP-SnT
(SEQ ID NO: 13)
MNNNDLFQASRRRFLAQLGGLTVAGMLGPSLLTPRRASMNNNDLFQAS
RRRFLAQLGGLTVAGMLGPSLLTPRRASMNNNDLFQASRRRFLAQLGG
LTVAGMLGPSLLTPRRASAKIEEGKLVIWINGDKGYNGLAEVGKKFEK
DTGIKVTVEHPDKLEEKFPQVAATGDGPDIIFWAHDRFGGYAQSGLLA
EITPDKAFQDKLYPFTWDAVRYNGKLIAYPIAVEALSLIYNKDLLPNP
PKTWEEIPALDKELKAKGKSALMFNLQEPYFTWPLIAADGGYAFKYEN
GKYDIKDVGVDNAGAKAGLTFLVDLIKNKHMNADTDYSIAEAAFNKGE
TAMTINGPWAWSNIDTSKVNYGVTVLPTFKGQPSKPFVGVLSAGINAA
SPNKELAKEFLENYLLTDEGLEAVNKDKPLGAVALKSYEEELAKDPRI
AATMENAQKGEIMPNIPQMSAFVVYAVRTAVINAASGRQTVDEALKDA
QTRITKGSGSGSKLGDIEFIKVNK*

Unless the context requires otherwise, the terms “comprise”, “comprises” and “comprising” or similar are intended to mean a non-exclusive inclusion, such that a recited list of elements or features does not include those stated or listed elements solely, but may include other elements or features that are not listed or stated.

While the invention has been described with reference to various exemplary aspects and embodiments, it will be understood by those skilled in the art that various changes may be made and equivalents may be substituted for elements thereof without departing from the broad spirit and scope of the invention. In addition, many modifications may be made to adapt a particular situation or molecule to the teachings of the invention without departing from the essential scope thereof. Therefore, it is intended that the invention not be limited to any particular embodiment contemplated, but that the invention will include all embodiments falling within the scope of the appended claims.

### Itemized List of Embodiments

1. An inclusion body comprising a coupling peptide suitable for coupling to a partner peptide through the formation of a covalent isopeptide bond.

2. The inclusion body according to item 1, wherein said coupling peptide comprises one residue involved in said isopeptide bond and said partner peptide comprises the other residue involved in said isopeptide bond.

3. The inclusion body according to any one of the preceding items, wherein when said coupling peptide comprises a reactive lysine residue, said partner peptide comprises a reactive asparagine, aspartic acid, glutamine or glutamic acid residue, or when said coupling peptide comprises a reactive asparagine, aspartic acid, glutamine or glutamic acid residue, said partner peptide comprises a reactive lysine residue or a reactive alpha-amino terminus.

4. The inclusion body according to any one of the preceding items, wherein said coupling peptide comprises a reactive asparagine residue and said partner peptide comprises a reactive lysine residue, or said coupling peptide comprises a reactive lysine residue and said partner peptide comprises a reactive asparagine residue.

5. The inclusion body according to any one of the preceding items, wherein said coupling peptide and partner peptide are derived from a protein of a Gram positive or Gram negative bacterium

6. The inclusion body according to item 5, wherein said protein is of a Gram positive bacterium.

7. The inclusion body according to item 5 or 6, wherein said protein is of a Gram positive bacterium from the Streptococcaceae family selected from Streptococcus pyogenes, Streptococcus pneumoniae or Streptococcus dysgalactiae.

8. The inclusion body according to any one of items 5-7, wherein said protein is adhesin RrgA of Streptococcus pneumoniae, fibronectin-binding protein FbaB of Streptococcus pyogenes, major pilin protein Spy0128 of Streptococcus pyogenes, or fibronectin-binding protein CnaB of Streptococcus dysgalactiae, or a protein with at least 70% sequence identity thereto which is capable of forming one or more isopeptide bonds.

9. The inclusion body according to any one of the preceding items, wherein the coupling peptide is selected from the group consisting of SpyTag, KTag, SnoopTag, SpyTag002, SpyTag003, SpyTag0128, SdyTag, DogTag, SnoopTagJr and BDTag.

10. The inclusion body according to any one of the preceding items, wherein the partner peptide is selected from the group consisting of SpyTag, KTag, SpyCatcher, SnoopCatcher, SpyCatcher002, SpyCatcher003, SpyCatcher0128, SdyCatcher, DogTag, SnoopTagJr and BDTag.

11. The inclusion body according to any one of items 1-8, wherein the coupling peptide and partner peptide form a ligation pair selected from the group consisting of SpyTag-SpyCatcher, SpyTag-SpyCatcher002, SnoopTag-SnoopCatcher, SpyTag002-SpyCatcher002, SpyTag002-SpyCatcher, SpyTag003-SpyCatcher003, SpyTag0128-SpyCatcher0128, SdyTag-SdyCatcher, KTag-SpyTag, SpyTag-KTag, DogTag-SnoopTagJr, SnoopTagJr-DogTag, SpyTag-BDTag and BDTag-SpyTag.

12. The inclusion body according to any one of items 1-8, wherein (i) the coupling peptide is KTag, the partner peptide is SpyTag and the formation of a covalent isopeptide bond is mediated by addition of SpyLigase; (ii) the coupling peptide is KTag, the partner peptide is SpyTag002 and the formation of a covalent isopeptide bond is mediated by addition of SpyLigase; (iii) the coupling peptide is SpyTag, the partner peptide is KTag and the formation of a covalent isopeptide bond is mediated by addition of SpyLigase; (iv) the coupling peptide is SpyTag002, the partner peptide is KTag and the formation of a covalent isopeptide bond is mediated by addition of SpyLigase; (v) the coupling peptide is DogTag, the partner peptide is SnoopTagJr and the formation of a covalent isopeptide bond is mediated by addition of SnoopLigase; (vi) the coupling peptide is SnoopTagJr, the partner peptide is DogTag and the formation of a covalent isopeptide bond is mediated by addition of SnoopLigase; (vii) the coupling peptide is SpyTag, the partner peptide is BDTag and the formation of a covalent isopeptide bond is mediated by addition of SpyStapler; or (viii) the coupling peptide is BDTag, the partner peptide is SpyTag and the formation of a covalent isopeptide bond is mediated by addition of SpyStapler.

13. A complex comprising the inclusion body according to any one of the preceding items coupled to the partner peptide via a covalent isopeptide bond between the coupling peptide and the partner peptide.

14. The inclusion body or complex according to any one of the preceding items, further comprising at least one protein of interest (POI) or a portion thereof.

15. The inclusion body or complex according to item 14, wherein said protein of interest is a protein with a therapeutic purpose that treats a condition or disorder selected from the group consisting of cancer, autoimmune disease, inflammatory disease, transplant rejection and infectious disease.

16. The inclusion body or complex according to item 14, wherein said protein of interest is a protein with a prophylactic purpose that protects against a condition or disorder selected from the group consisting of cancer, autoimmune disease, inflammatory disease, transplant rejection and infectious disease.

17. The inclusion body or complex according to item 14, wherein said protein of interest or portion thereof is an antigen or fragment thereof.

18. The inclusion body or complex according to item 17, wherein said antigen is selected from the group consisting of an antigen from an infectious organism, a tumor antigen, a tumor stroma antigen or a tumor associated antigen.

19. The inclusion body according to any one of the preceding items, further comprising an inclusion body forming sequence.

20. The inclusion body or complex according to any one of the preceding items, wherein the partner peptide comprises an additional moiety.

21. The inclusion body or complex according to item 20, wherein the additional moiety is selected from the group consisting of a glycan, an adjuvant, an adhesion molecule, an enzyme and a traceable probe.

22. The inclusion body or complex according to item 20, wherein the additional moiety is an immune modulating compound.

23. The inclusion body or complex according to item 22, wherein the immune modulating compound is selected from the group consisting of a cytokine, an adjuvant, an antibody, Nanobody® molecule, a DARPIN, PAMP, a TLR ligand or agonist, RNA, DNA, an immunomodulating peptide, a peptidomimetic, a T helper cell epitope, an immune checkpoint inhibitor, PLGA, chitosan and TRAIL.

24. The inclusion body or complex according to item 20, wherein the additional moiety is a targeting moiety.

25. The inclusion body or complex according to item 24, wherein the targeting moiety has an affinity fora cell of the immune system.

26. The inclusion body or complex according to item 25, wherein the targeting moiety has an affinity for a surface exposed component of a cell of the immune system

27. The inclusion body or complex according to item 26, wherein the surface exposed component is selected from the group consisting of CD4, CD8, CD1, CD180, IgA, IgD, IgE, IgG, IgM, TCR, CRDs, Toll-like receptors (TLRs), nucleotide-binding oligomerization domain-like receptors (NLRs), retinoic acid-inducible gene I-like helicases receptors (RLRs), and C-type lectin receptors (CLRs), endocytic receptors, CD205/DEC205, CD209/DC-SIGN, Clec9A/DNGR-1/CD370, Clec7A/Dectin-1/CD369, Clec6A/Dectin-2, Clec12A, CD1d, CD11c, CD11 b, CD40, CD152/CTLA-4, CD279/PD-1, NOD-like receptors, RIG-I-like receptors, PRRs, CCRs, CD36, Siglec H, PDCTREM, Langerin, MMR, D-SIGN and Folate receptors.

28. The inclusion body or complex according to item 24, wherein the targeting moiety has an affinity for at least one diseased cell.

29. The inclusion body or complex according to item 28, wherein the diseased cell is a tumor cell of a cancer.

30. The inclusion body or complex according to item 29, wherein the cancer is selected from the group consisting of lymphoma, leukemia, myeloma, lung cancer, melanoma, renal cell cancer, ovarian cancer, glioblastoma, Merkel cell carcinoma, bladder cancer, head and neck cancer, colorectal cancer, esophageal cancer, cervical cancer, gastric cancer, hepatocellular cancer, prostate cancer, breast cancer, pancreatic cancer and thyroid cancer.

31. The inclusion body or complex according to any one of items 24-30, wherein the targeting moiety is an antibody, an antibody domain or an antibody fragment retaining antibody binding.

32. A nucleic acid encoding the inclusion body forming polypeptide of the inclusion body according to any one of the preceding items.

33. A genetic construct comprising the nucleic acid according to item 32.

34. A host cell comprising the nucleic acid according to item 32 or the genetic construct according to item 33.

35. A composition comprising the inclusion body, complex, nucleic acid, genetic construct and/or host cell according to any one of the preceding items.

36. An inclusion body, complex or composition according to any one of the preceding items for use as a medicament.

37. An inclusion body, complex or composition according to any one of the preceding items for use as a diagnostic, prognostic, prophylactic or therapeutic agent.

38. An inclusion body, complex or composition according to any one of the preceding items for use as a vaccine.

39. A method of treatment of a disease or disorder in a subject comprising the step of introducing the inclusion body, complex or composition according to any one of the preceding items to said subject.

40. A method of diagnosis or prognosis of a disease or disorder in a subject using the inclusion body, complex or composition according to any one of items 1-35.

41. A method of vaccination or immunization comprising the step of introducing the inclusion body, complex or composition according to any one of items 1-35 to said subject.

42. The method according to any one of items 39-41, wherein said subject is an animal.

43. The method according to item 42, wherein said animal is a mammal.

44. The method according to item 43, wherein said mammal is a human, a farm animal or a companion animal.

45. The method according to item 42, wherein said animal is a bird or a fish.

46. A method of producing the complex according to embodiment 13, comprising the step of conjugating the inclusion body of embodiment 1 to a partner peptide to thereby produce said complex.

47. The method according to embodiment 46, wherein the complex is produced by the formation of a covalent isopeptide bond between the coupling peptide of the inclusion body and the partner peptide.

48. The method according to embodiment 47, wherein the coupling peptide comprises one residue involved in said isopeptide bond and said partner peptide comprises the other residue involved in said isopeptide bond.

49. The method according to any one of embodiments 46-48, wherein the inclusion body is conjugated to a partner peptide presented in a lysate, such as a cell lysate, such as a bacterial lysate.

50. The method according to any one of embodiments 46-48, wherein the inclusion body is conjugated to a purified partner peptide.

51. The method according to any one of embodiments 46-50, wherein the coupling peptide is selected from the group consisting of SpyTag, KTag, SnoopTag, SpyTag002, SpyTag003, SpyTag0128, SdyTag, DogTag, SnoopTagJr and BDTag.

52. The method according to any one of embodiments 46-51, wherein the partner peptide is selected from the group consisting of SpyTag, KTag, SpyCatcher, SnoopCatcher, SpyCatcher002, SpyCatcher003, SpyCatcher0128, SdyCatcher, DogTag, SnoopTagJr and BDTag.

53. The method according to any one of embodiments 46-52, wherein the complex is produced following the formation of a coupling peptide-partner peptide ligation pair selected from the group consisting of SpyTag-SpyCatcher, SpyTag-SpyCatcher002, SnoopTag-SnoopCatcher, SpyTag002-SpyCatcher002, SpyTag002-SpyCatcher, SpyTag003-SpyCatcher003, SpyTag0128-SpyCatcher0128, SdyTag-SdyCatcher, KTag-SpyTag, SpyTag-KTag, DogTag-SnoopTagJr, SnoopTagJr-DogTag, SpyTag-BDTag and BDTag-SpyTag.

1. An inclusion body comprising a coupling peptide suitable for coupling to a partner peptide through the formation of a covalent isopeptide bond.

2. The inclusion body according to claim 1, wherein said coupling peptide comprises one residue involved in said isopeptide bond and said partner peptide comprises the other residue involved in said isopeptide bond.

3. The inclusion body according to claim 1, wherein said coupling peptide and partner peptide are derived from a protein of a Gram positive or Gram negative bacterium

4. The inclusion body according to claim 1, wherein the coupling peptide is selected from the group consisting of SpyTag, KTag, SnoopTag, SpyTag002, SpyTag003, SpyTag0128, SdyTag, DogTag, SnoopTagJr and BDTag.

5. The inclusion body according to claim 1, wherein the partner peptide is selected from the group consisting of SpyTag, KTag, SpyCatcher, SnoopCatcher, SpyCatcher002, SpyCatcher003, SpyCatcher0128, SdyCatcher, DogTag, SnoopTagJr and BDTag.

6. The inclusion body according to claim 1, wherein the coupling peptide and partner peptide form a ligation pair selected from the group consisting of SpyTag-SpyCatcher, SpyTag-SpyCatcher002, SnoopTag-SnoopCatcher, SpyTag002-SpyCatcher002, SpyTag002-SpyCatcher, SpyTag003-SpyCatcher003, SpyTag0128-SpyCatcher0128, SdyTag-SdyCatcher, KTag-SpyTag, SpyTag-KTag, DogTag-SnoopTagJr, SnoopTagJr-DogTag, SpyTag-BDTag and BDTag-SpyTag.

7. A complex comprising the inclusion body according to claim 1 coupled to the partner peptide via a covalent isopeptide bond between the coupling peptide and the partner peptide.

8. The inclusion body according to claim 1, further comprising at least one protein of interest (POI) or a portion thereof.

9. The inclusion body according to claim 8, wherein said protein of interest or portion thereof is an antigen or fragment thereof.

10. The inclusion body according to claim 1, further comprising an inclusion body forming sequence.

11. The inclusion body according to claim 1, wherein the partner peptide comprises an additional moiety.

12. The inclusion body according to claim 11, wherein the additional moiety is a targeting moiety.

13. A nucleic acid encoding the inclusion body forming polypeptide of the inclusion body according to claim 1.

14. A composition comprising the inclusion body according to claim 1.

15. A method of treating, diagnosing, prognosing, or preventing a disease or disorder in a subject, comprising administering the inclusion body according to claim 1 as a diagnostic, prognostic, prophylactic or therapeutic agent to a subject in need thereof.

16. A method of producing the complex according to claim 7, comprising the step of conjugating the inclusion body to a partner peptide to thereby produce said complex.

17-23. (canceled)

24. The complex according to claim 7, further comprising at least one protein of interest (POI) or a portion thereof.

25. The complex according to claim 24, wherein said protein of interest or portion thereof is an antigen or fragment thereof.

26. The complex according to claim 7, wherein the partner peptide comprises an additional moiety.

27. The complex according to claim 26, wherein the additional moiety is a targeting moiety.

28. A composition comprising the complex according to claim 7.

29. A method of treating, diagnosing, prognosing, or preventing a disease or disorder in a subject, comprising administering the complex according to claim 7 as a diagnostic, prognostic, prophylactic, or therapeutic agent to a subject in need thereof.
